(19) United States
(12) Patent Application Publication
(10) Pub. No.: US 2023/0414720 A1
(43) Pub. Date: Dec. 28, 2023

(54) USE OF INSULIN-LIKE GROWTH FACTORS WITH GAMMA-CHAIN CYTOKINES TO INDUCE HOMEOSTATIC PROLIFERATION OF LYMPHOCYTES

(71) Applicant: UNIVERSITY OF FLORIDA RESEARCH FOUNDATION, INCORPORATED, Gainesville, FL (US)

(72) Inventors: Todd Michael BRUSKO, Gainesville, FL (US); Melanie SHAPIRO, Gainesville, FL (US)

(21) Appl. No.: 18/038,321

(22) PCT Filed: Nov. 23, 2021
(86) PCT No.: PCT/US2021/060574

§ 371 Date: May 23, 2023

Related U.S. Application Data
(60) Provisional application No. 63/117,081, filed on Nov. 23, 2020.

(51) Int. Cl. A61K 38/20; C12N 5/0783; A61K 38/30; C07K 16/24; A61K 35/17

(52) U.S. Cl. CPC A61K 38/2013; C12N 5/0637; A61K 38/30; C07K 16/246; A61K 35/17; C12N 2501/105; C12N 2501/2302; C12N 2510/00

(57) ABSTRACT

Low-dose IL-2 has been proposed as a therapy to rectify regulatory T cell (Treg) fitness in type 1 diabetes (T1D). Identification of a combinatorial approach permits lower IL-2 dosing and avoids off-target effects. Insulin-like growth factor-1 (IGF1) is a hormone that promotes immunoregulation. Disclosed are methods that synergizes IGF1 with IL-2 to expand Tregs. In vivo IL-2+IGF1 treatment expands murine Tregs to a greater extent than either agent alone. IGF1+IL-2 treatment permits gene transfer in primary human Treg without the requirement for conventional activation stimuli, observing that IGF1+IL-2 maintains Treg naivety.

### GOVERNMENT SUPPORT

This invention was made with government support under Grant Nos. AI042288 and DK117548 awarded by the National Institutes of Health. The government has certain rights in the invention.

### BACKGROUND

Impairments in immunoregulation, including defective regulatory T cell (Treg) generation in the thymus as well as peripheral maintenance of phenotypically stable and functional Treg populations, have been suggested to contribute to the development of type 1 diabetes (T1D) and other autoimmune diseases (1,2). Recent thymic emigrant tracking experiments have revealed that non-obese diabetic (NOD) mice export a decreased percentage of Tregs from the thymus as compared to non-autoimmune C57BL/6 mice, leading to an imbalance of Treg to effector T cells (Teff) prior to disease onset (3). Although human studies have largely shown that Treg numbers are normal in the peripheral blood of T1D subjects (4-6), evidence suggests maintenance of Tregs to be compromised in pancreatic draining lymph nodes of human organ donors with T1D (7). Efforts to specifically increase Treg without influencing Teff numbers are therefore of great interest for T1D prevention.

Low-dose IL-2 has been proposed as a means to selectively enhance the proliferation and function of Tregs, which constitutively express CD25, the alpha chain subunit of IL-2 receptor (IL-2Rα) that creates the high-affinity trimeric receptor. However, inflammatory cell subsets expressing CD25, such as memory T cells and natural killer (NK) cells, have been reported to expand alongside Tregs with IL-2 administration (8,9). This highlights the need for identification of combination therapies that synergize with IL-2 to allow for efficacy with lower IL-2 doses and avoidance of off-target effects. Since CD25 expression is lower on naïve than memory Tregs, the former are much less responsive to low-dose IL-2 therapy (8), and naïve Treg show limited proliferative capacity in vivo (10,11). However, naïve Tregs show superior maintenance of phenotypic stability (12,13) and survival (14) as compared to memory Tregs and are thus a favorable target for expanding the overall Treg population. Therefore, additional cytokines or growth factors capable of promoting naïve CD4⁺ T cell proliferation may be required to enhance the impact of low-dose IL-2 on naïve Tregs specifically.

### BRIEF DESCRIPTION OF THE DRAWINGS

The present embodiments are illustrated by way of example, and not by way of limitation, in the figures of the accompanying drawings.

The following figures are illustrative only, and are not intended to be limiting.

FIG. 1A are flow cytometry graphs showing gating strategy for IGF1R expression analysis. Whole blood staining was performed to gate CD3⁺CD4⁺ T cells into naïve (CD45RA⁺CD197⁺), memory (CD45RA⁻), conventional (CD127⁺), and regulatory (CD25^{hi}CD127^{lo/-}) subsets.

FIG. 1B-C are representative histograms comparing marker expressions in different T cell populations. The histogram shows geometric mean fluorescence intensity (gMFI) of CD221/IGF1R on naïve Treg (green), naïve Tconv (orange), memory Tconv (pink), and memory Treg (blue) within one subject. CD221/IGF1R expression is highest on naïve Treg, followed closely by naïve Tconv. Memory Tconv and memory Treg show lowest CD221/IGF1R expression. Friedman test with Dunn's multiple comparisons test: *, p<0.05; **, p<0.01; ***, p<0.001; ****, p<0.0001.

FIG. 2 is the comparison of IGF1R expression on CD4⁺ T cell subsets of T1D subjects and relatives. Whole blood staining revealed that CD221/IGF1R geometric mean fluorescence intensity (gMFI) was not significantly different within naïve Tconv (orange), memory Tconv (pink), naïve Treg (green), memory Treg (blue) subsets when comparing patients with T1D and relatives (rel). Mann-Whitney test.

FIG. 3A-D are plots showing IGF1R expression on CD4⁺ T cells decrease with age of subject. Whole blood staining revealed a significant negative correlation between CD221/IGF1R gMFI and age of subject in years for A) naïve Tconv, B) naïve Treg, and D) memory Treg. A trend for decreased CD221/IGF1R with age was noted in C) memory Tconv. Best fit lines shown (solid line, blue) with 95% confidence intervals (dashed lines, blue). Spearman correlation R and p values shown in upper right-hand corner of plot.

FIG. 4A is the Gating strategy for naïve (CD45RA⁺) and memory (CD45RA⁻) CD4⁺ T cells.

FIG. 4B-E are histograms and plots showing IGF1 preferentially signals to naïve versus memory CD4⁺ T cells. B) Representative histogram of naïve and memory expression of pAkt (Ser473) at baseline (red) and 15 minutes of IGF1 treatment (turquoise). C) Fold change of pAkt gMFI was higher in naïve than memory CD4⁺ T cells at 15 and 30 minutes of IGF1. D) Representative histogram of pS6 (Ser235/236) expression at baseline and 15 minutes of IGF1 treatment. E) Fold change in percent of pS6+ cells was higher in naïve than memory CD4+ T cells at 15 and 30 minutes of IGF1 treatment. Wilcoxon-test: *, p<0.05; **, p<0.01.

FIG. 5A is the continued strategy for gating naïve CD4⁺ T cells into Tconv (FOXP3⁻Helios⁻) and Treg (FOXP3⁺Helios⁺) subsets.

FIG. 5B-C are plots showing IGF1 induces comparable PI3K/Akt signaling in naïve Tconv and naïve Treg. No significant differences were observed when comparing naïve Tconv to naïve Treg for fold change of B) pAkt gMFI or C) percent of pS6 positivity at 15 minutes and 30 minutes of IGF1 treatment. Wilcoxon-test.

FIG. 6A is the method for in vitro naïve CD4⁺ T cell homeostatic proliferation experiments.

FIG. 6B-F are plots showing that IGF1 augments the IL-2-mediated homeostatic proliferation of naïve Treg. B) Representative plot of FOXP3 and Helios expression on CD4⁺CD45RA⁺ T cells showing that C) percentage of FOXP3⁺Helios⁺ cells was increased upon treatment with IL-2+IGF1 (turquoise) as compared to IL-2 alone (red). Paired t test: **, p=Representative plots of cell proliferation dye show D) no proliferation of naïve Tconv in either condition, or E) enhanced proliferation of naïve Treg in the presence of IL-2+IGF1 versus IL-2 alone, F) as quantified by the division index. Wilcoxon-test.

FIG. 7 are plots showing that IGF1 synergizes with LD IL-2 to promote PI3K/Akt signaling specifically in Tregs of NOD mice. Tregs were defined as Foxp3⁺Helios⁺. Data were normalized by % pS6⁺ without treatment/mouse. Repeated measures two-way ANOVA with Tukey's multiple comparisons test.

FIG. 8A is an experimental scheme for IGF1 and IL-2 treatments to 12-week-old prediabetic NOD mice.

FIG. 8B-F are dot plots and representative histograms showing IGF1 treatment stimulates Tregs and synergizes with IL-2. B) Representative dot plots showing CD8 & CD4, Foxp3 & CD25, and CD335 & CD122 expression in the indicated gated+ population. C) representative histogram showing Ki67 expression in CD8, CD4+Foxp3−, Treg and CD335+ cells, and D) Representative dot plot showing Helios & Ki67 in gated Tregs 1-week after the start of treatment. E) The percentage of CD4+Foxp3+ cells over day 0 in the peripheral blood 1, 2, and 3 weeks after the start of treatments, n=4 mice per groups. One-way ANOVA with E) Dunnett's multiple comparison to the PBS control or F) Tukey's multiple comparison at each timepoint.

FIG. 9A-C are plots showing that IGF1 treatment induces Treg proliferation. The plots show percentage of Ki67+in A) total CD4+Foxp3+ Tregs, B) Helios+ Tregs, or C) Helios− Tregs. One-way ANOVA with Dunnett's multiple comparison to the PBS control at each timepoint.

FIG. 10A-D shows murine IGF1R expression across the maturation stages of Tconv and Treg. Thymus and spleen of pre-diabetic NOD.Foxp3-GFP mice were stained for IGF1R and differentiation of CD4⁺ and CD8⁺ naïve and memory T cells via flow cytometry. (A) Representative histogram of IGF1R/CD221 expression on CD4⁻CD8⁻ double negative (DN), CD4⁺CD8⁺ double positive (DP), CD4⁺CD8⁻ single positive (CD4 SP), and CD4⁻CD8⁺ (CD8 SP) thymocytes. (B) Geometric mean fluorescence intensity (gMFI) of IGF1R at various stages of thymocyte development. (C) Representative histogram of IGF1R expression on naïve (CD62L⁺CD44^{lo}) and memory (CD62L⁻CD44^{hu}) Tconv (CD4⁺Foxp3⁻) and Treg (CD4⁺Foxp3⁺) populations in spleen. (D) gMFI of IGF1R on CD4⁺ T cell subpopulations.

FIG. 11A-F shows IGF1 synergizes with LD IL-2 to promote PI3K/Akt signaling specifically in Tregs of NOD mice. Splenocytes of pre-diabetic mice (14-22 weeks old) were stimulated with IGF1 (100 ng/mL, n=3, purple), IL-2 (10 IU/mL, n=5, blue), or IGF1+IL-2 (n=5, green) for 15 or 60 minutes prior to phosflow staining. (A) Representative contour plots of pS6 (Ser235/236) induction in CD44⁻ Tconv (CD4⁺Foxp3⁻Helios⁻), CD44⁺ Tconv, and Treg (CD4⁺Foxp3⁺Helios⁺). (B) Data were normalized by % pS6⁺ cells without treatment/mouse to calculate fold change. Repeated measures two-way ANOVA with Tukey's multiple comparisons test. CD44⁻ Tconv: Time×Treatment, p=0.238; Time, p=0.004; Treatment, p=0.091. CD44⁺ Tconv: Time×Treatment, p=0.234; Time, p=0.003; Treatment, p=0.201. Treg: Time×Treatment, p=0.032; Time, p=0.003; Treatment, p=0.033. (C) Representative histograms of pStat5 (Tyr694) induction in CD44⁻ Tconv, CD44⁺ Tconv, and Treg with IL-2 titration (n=2; gray=no treatment, periwinkle=1 IU/mL, light blue=10 IU/mL, blue=100 IU/mL IL-2). (D) Data were normalized by pStat5 gMFI without treatment/mouse to calculate fold change. (E) Representative histograms of pStat5 (Tyr694) induction in CD44⁻ Tconv, CD44⁺ Tconv, and Treg without treatment (gray), with 10 IU/mL IL-2 alone (blue), or 10 IU/mL IL-2+100 ng/mL IGF1 (green); n=2. (F) Data were normalized by pStat5 gMFI without treatment/mouse to calculate fold change.

FIG. 12A-E shows IGF1 treatment stimulates Tregs and synergizes with IL-2. (A) Experimental scheme. Prediabetic NOD mice were treated with PBS, IL-2 antibody complex [IAC: anti-IL-2 (JES6.1)+rmIL-2], IGF1, or IGF1+IAC starting at 12 weeks of age. IL-2 complex was delivered every other day for 7 days, while IGF-1 was delivered as 10 ug subcutaneous injections twice a day for a duration of 3 weeks. Peripheral blood was collected for flow cytometric analysis before treatment and weekly for up to 3 weeks post-treatment. (B) Representative dot plots showing CD4⁺Foxp3⁺CD25⁺ Treg and CD4⁺Foxp3⁻ Tconv in peripheral blood after one week of treatment. (C) Percentage of CD4⁺Foxp3⁺CD25⁺ Treg at 0, 7, 14, and 21 days in PBS (white), IAC (blue), IGF1 (purple), and IGF1+IAC (green) treated groups. One-way ANOVA with Dunnett's multiple comparison to the PBS timepoint. Fold change of Treg over day 0 in the peripheral blood 1, 2, and 3 weeks after the start of treatments. One-way ANOVA with Tukey's multiple comparison at each timepoint. Plots were pre-gated on CD45⁺CD19⁻Ly6G⁻ cells. (D) Representative dot plots showing Helios and Ki67 expression on CD4⁺Foxp3⁺ Tregs in peripheral blood after one week of treatment. (E) Percentage of Ki67⁺ cells in Helios⁺ Tregs or Helios⁻ Tregs. One-way ANOVA with Dunnett's multiple comparison to the PBS timepoint. n=4 mice per group. ns=not significant.

FIG. 13A-E shows naive human Treg express significantly higher levels of IGF1R than naïve Tconv. Whole blood staining was performed to gate CD3⁺CD4⁺ T cells into naïve (CD45RA⁺CD197⁺), memory (CD45RA⁻), conventional (CD127⁺), and regulatory (CD25^{hi}CD127^{lo/-}) subsets. (A) Representative histogram of geometric mean fluorescence intensity (gMFI) of IGF1R (CD221) on naïve Treg (green), naïve Tconv (orange), memory Tconv (pink), and memory Treg (blue) within one subject. (B) IGF1R expression is highest on naïve Treg, followed closely by naïve Tconv. Memory Tconv and memory Treg show lowest IGF1R expression. Friedman test with Dunn's multiple comparisons test. Whole blood staining revealed a negative correlation between IGF1R gMFI and age of subject in years for (C) naïve and memory Tconv and (D) naïve and memory Treg subsets. Best fit third-order polynomial function shown (solid line, blue) with 95% confidence intervals (dashed lines, blue). Spearman correlation R and p values shown below. (E) Whole blood staining revealed that IGF1R gMFI was not significantly different within naïve Tconv, memory Tconv, naïve Treg, memory Treg subsets when comparing patients with T1D and relatives (rel). Mann-Whitney test. n=29 subjects.

FIG. 14A-F shows IGF1 augments the IL-2-mediated homeostatic proliferation of naïve Treg. (A) Methods for in vitro naïve CD4⁺ T cell homeostatic proliferation experiments. Density gradient centrifugation was performed to isolate PBMCs from whole blood, followed by magnetic bead-based naïve CD4⁺ T cell enrichment. Cells were stained with Cell Proliferation Dye eFluor670 to track proliferation prior to plating in complete RPMI supplemented with 20 IU/mL IL-2 and/or 100 ng/mL IGF1. Cytokines were replenished on days 3 and 7 and cultures were stained for flow cytometric analysis on day 9-11. Image created with BioRender. (B) Representative plot of FOXP3 and Helios expression on CD4⁺CD45RA⁺ T cells showing that (C) percentage of FOXP3⁺Helios⁺ cells was increased upon treatment with IL-2+IGF1 (turquoise) as compared to IL-2 alone (red). Paired t test. Representative plots of cell proliferation dye show (D) no proliferation of naïve Tconv in either condition, or (E) enhanced proliferation of naïve Treg in the presence of IL-2+IGF1 versus IL-2 alone, (F) as quantified by the division index. Wilcoxon-test. n=5 subjects.

FIG. 15A-C shows IGF1 permits lentiviral-mediated transduction while maintaining T cell naivety. Naïve Treg or Tconv were sorted and cultured with 20 IU/mL IL-2, with or without 100 ng/mL IGF1, followed by transduction with lentiviral constructs containing T1D-related β-cell-specific T cell receptor (TCR) R164, recognizing glutamic acid decarboxylase (GAD) 555-567 in the context of human leukocyte antigen (HLA)-DRB1*04:01. Plots are pre-gated on antigen-specific cells. (A) Dot plot of CD45RA and CD197 showing that cells remained a naïve phenotype post-transduction. (B) Dye dilution assay shows enhanced proliferation of naïve Treg with IL-2+IGF1 (red) as compared to IL-2 (blue) alone. Proliferation index (PI) noted adjacent to histogram per condition. (C) Dot plot of TRBV5-1 expression showing successful lentiviral transduction. n=2 subjects.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference.

Generally, nomenclatures used in connection with, and techniques of, cell and tissue culture, molecular biology, immunology, microbiology, genetics, protein, and nucleic acid chemistry and hybridization described herein are those well-known and commonly used in the art. The methods and techniques of the present invention are generally performed according to conventional methods well known in the art and as described in various general and more specific references that are cited and discussed through the present specification unless otherwise indicated.

The term “about” means plus or minus 1%, 2%, 3%, 4%, 5%, 6%, 7%, 8%, 9%, or 10% of the number to which reference is being made.

The term “cell proliferation” as used herein refers to the process by which a cell grows and divides to produce two daughter cells. Cell proliferation leads to an exponential increase in cell number.

The terms “culture”, “cultured”, or “culturing” as used herein refers to the process by which cells are grown under controlled conditions, generally outside their natural environment. After the cells of interest have been isolated from living tissue, they can subsequently be maintained under carefully controlled conditions and methods known in the arts. Maintenance of the cells comprises of passaging the cells and replenishing growth factors and nutrients.

The terms “growth factor” as used herein refers to a naturally occurring substance capable of stimulating cell proliferation, wound healing, and occasionally cellular differentiation. Usually it is a protein or a steroid hormone, such as interferon, interleukin, or cytokine, which are secreted by certain cells of the immune system and have an effect on other cells.

The term “homeostatic” as used herein refers to cells in a steady-state condition and are in a stable non-differentiated state. Cells undergoing homeostatic proliferation do not experience a change in cellular differentiation, maturation, or activation while growing or dividing.

The term “immune cell” as used herein refers to cells that play a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes. For example, antigen-reactive T cells are T cells that selectively bind to an antigen of interest and modulate immunological responses based upon the recognition of antigen.

The terms “Insulin-like growth factor 1” or “IGF1” as used herein refers to a protein that in humans is encoded by the IGF1 gene. IGF1 consists of 70 amino acids in a single chain with three intramolecular disulfide bridges. IGF1 has a molecular weight of 7,649 Daltons. IGF1 is a hormone similar in molecular structure to insulin which plays an important role in childhood growth and has anabolic effects in adults.

The term “common gamma chain” refers to a cytokine receptor sub-unit that is common to the receptor complexes for at least six different interleukin receptors: IL-2, IL-4, IL-7, IL-9, IL-15 and interleukin-21 receptor. It is also known as γ_{c}, CD132, interleukin-2 receptor subunit gamma, or IL-2RG. The common gamma chain is a member of the type I cytokine receptor family expressed on most lymphocyte (white blood cell) populations, and its gene is found on the X-chromosome of mammals. This protein is located on the surface of immature blood-forming cells in bone marrow. One end of the protein resides outside the cell where it binds to cytokines and the other end of the protein resides in the interior of the cell where it transmits signals to the cell's nucleus. The common gamma chain partners with other proteins to direct blood-forming cells to form lymphocytes (a type of white blood cell). The receptor also directs the growth and maturation of lymphocyte subtypes: T cells, B cells, and natural killer cells. These cells kill viruses, make antibodies, and help regulate the entire immune system.

The terms “Interleukin-2” or “IL-2” as used herein refers to a protein that in humans is encoded by the IL2 gene. IL-2 consists of 153 amino acids and has a molecular weight of 17,628 Daltons. IL-2 is a secreted cytokine that is important for the proliferation of T and B lymphocytes.

The term “naïve” as used herein refers to a T cell that has differentiated in bone marrow, and successfully undergone the positive and negative processes of central selection in the thymus and are mature, but have not been activated and are not memory cells. A naive T cell is considered immature and, unlike activated or memory T cells, has not encountered its cognate antigen within the periphery. Naïve T cells are commonly characterized by the surface expression of L-selectin (CD62L); the absence of the activation markers, CD25, CD44, or CD69; and the absence of memory CD45RO isoform. They also express functional IL-7 receptors, consisting of subunits IL-7 receptor-α, CD127, and common-γ chain, CD132. In the naive state, T cells are thought to be quiescent and non-dividing, requiring the common-gamma chain cytokines IL-7 and IL-15 for homeostatic survival mechanisms.

The term “obtained from a biological material source” as used herein means any conventional method of harvesting or partitioning a source of biological material from a donor. For example, biological material may obtained from a solid tumor, a blood sample, such as a peripheral or cord blood sample, or harvested from another body fluid, such as bone marrow or amniotic fluid. Methods for obtaining such samples are well-known to the artisan. In the present invention, the samples may be fresh (i.e., obtained from a donor without freezing). Moreover, the samples may be further manipulated to remove extraneous or unwanted components prior to expansion. The samples may also be obtained from a preserved stock. For example, in the case of cell lines or fluids, such as peripheral or cord blood, the samples may be withdrawn from a cryogenically or otherwise preserved bank of such cell lines or fluid. Such samples may be obtained from any suitable donor.

The term “T cell” as used herein includes CD4⁺ T cells and CD8⁺ T cells. The term T cell also includes both T helper 1 type T cells and T helper 2 type T cells. The term “antigen presenting cell” includes professional antigen presenting cells (e.g., B lymphocytes, monocytes, dendritic cells, Langerhans cells), as well as other antigen presenting cells (e.g., keratinocytes, endothelial cells, astrocytes, fibroblasts, and oligodendrocytes).

The terms “regulatory T cells” or “Tregs,” formerly known as “suppressor T cells,” as used herein refer to a subpopulation of T cells which modulate the immune system, maintain tolerance to self-antigens, and prevent autoimmune disease. Tregs may be distinguished based on expression of cell surface markers where Tregs generally have the phenotype: CD4⁺, CD25⁺, CD127^{low}, Foxp3⁺. In particular, Tregs that express CD4⁺ and Foxp3⁺ have been called “natural Tregs” or “nTregs” to distinguish them from “suppressor” T cell populations that are generated in vitro.

The term “subject” as used herein refers to an individual. For example, the subject is a mammal, such as a primate, and, more specifically, a human. The term does not denote a particular age or sex. Thus, adult and newborn subjects, whether male or female, are intended to be covered. As used herein, patient or subject may be used interchangeably and can refer to a subject afflicted with a disease or disorder.

The term “vector” as used herein refers to a nucleic acid capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” or simply “expression vectors”. In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions. The term “lentiviral vector” refers to a vector derived from (i.e., sharing nucleotides sequences unique to) a lentivirus.

The “CRISPR/Cas” system refers to a widespread class of bacterial systems for defense against foreign nucleic acid. CRISPR/Cas systems are found in a wide range of eubacterial and archaeal organisms. CRISPR/Cas systems include type I, II, and III sub-types. Wild-type type II CRISPR/Cas systems utilize an RNA-mediated nuclease, for example, Cas9, in complex with guide and activating RNA to recognize and cleave foreign nucleic acid. Guide RNAs having the activity of both a guide RNA and an activating RNA are also known in the art. In some cases, such dual activity guide RNAs are referred to as a single guide RNA (sgRNA).

Cas9 homologs are found in a wide variety of eubacteria, including, but not limited to bacteria of the following taxonomic groups: Actinobacteria, Aquificae, Bacteroidetes—Chlorobi, Chlamydiae—Verrucomicrobia, Chlroflexi, Cyanobacteria, Firmicutes, Proteobacteria, Spirochaetes, and Thermotogae. An exemplary Cas9 protein is the Streptococcus pyogenes Cas9 protein. Additional Cas9 proteins and homologs thereof are described in, e.g., Chylinksi, et al., RNA Biol. 2013 May 1; 10(5): 726-737; Nat. Rev. Microbiol. 2011 June; 9(6): 467-477; Hou, et al., Proc Natl Acad Sci U S A. 2013 Sep. 24;1 10(39): 15644-9; Sampson et al., Nature. 2013 May 9; 497(7448):254-7; and Jinek, et al., Science. 2012 Aug. 17; 337(6096):816-21. Variants of any of the Cas9 nucleases provided herein can be optimized for efficient activity or enhanced stability in the host cell. Thus, engineered Cas9 nucleases are also contemplated.

The term “guide RNA (gRNA) sequence” is a sequence that interacts with a site-specific or targeted nuclease and specifically binds to or hybridizes to a target nucleic acid within the genome of a cell, such that the gRNA and the targeted nuclease co-localize to the target nucleic acid in the genome of the cell. Each gRNA includes a DNA targeting sequence or protospacer sequence of about 10 to 50 nucleotides in length that specifically binds to or hybridizes to a target DNA sequence in the genome. For example, the targeting sequence may be about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the gRNA comprises a crRNA sequence and a transactivating crRNA (tracrRNA) sequence. In some embodiments, the gRNA does not comprise a tracrRNA sequence.

The term “Cas9” refers to an RNA-mediated nuclease (e.g., of bacterial or archeal origin, or derived therefrom). Exemplary RNA-mediated nucleases include the foregoing Cas9 proteins and homologs thereof. Other RNA-mediated nucleases include Cpfl (See, e.g., Zetsche et al., Cell, Volume 163, Issue 3, p′759-771, 22 Oct. 2015) and homologs thereof. Similarly, as used herein, the term“Cas9 ribonucleoprotein” complex and the like refers to a complex between the Cas9 protein and a guide RNA, the Cas9 protein and a crRNA, the Cas9 protein and a trans-activating crRNA (tracrRNA), or a combination thereof (e.g., a complex containing the Cas9 protein, a tracrRNA, and a crRNA guide RNA). It is understood that in any of the embodiments described herein, a Cas9 nuclease can be substituted with a Cpfl nuclease or any other guided nuclease.

### DETAILED DESCRIPTION

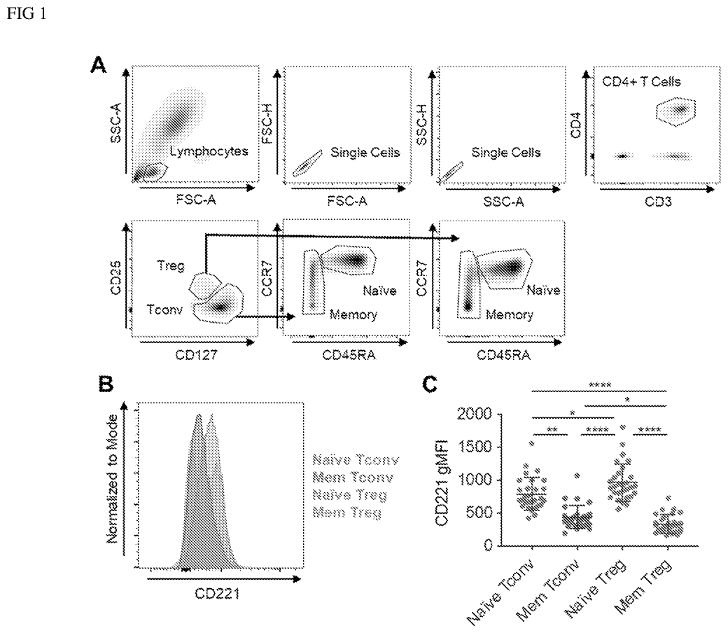

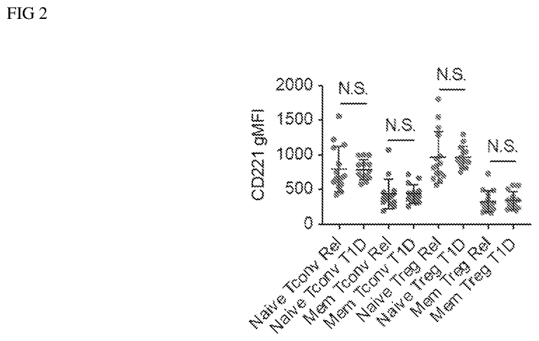

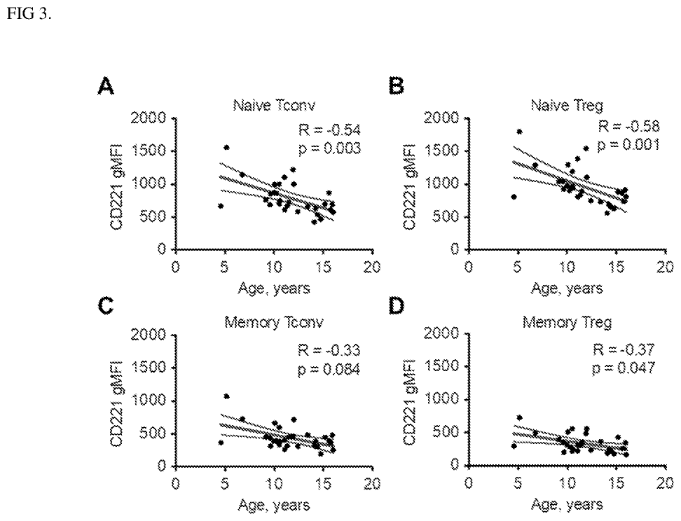

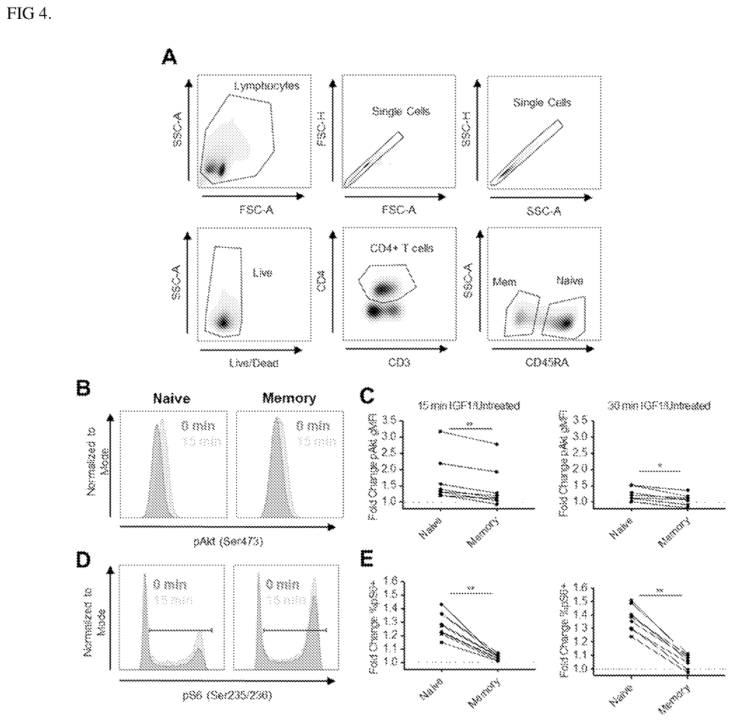

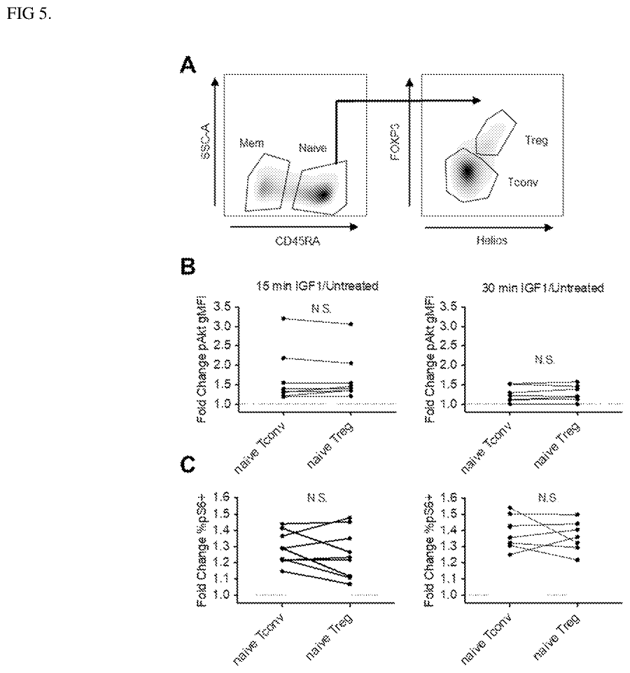

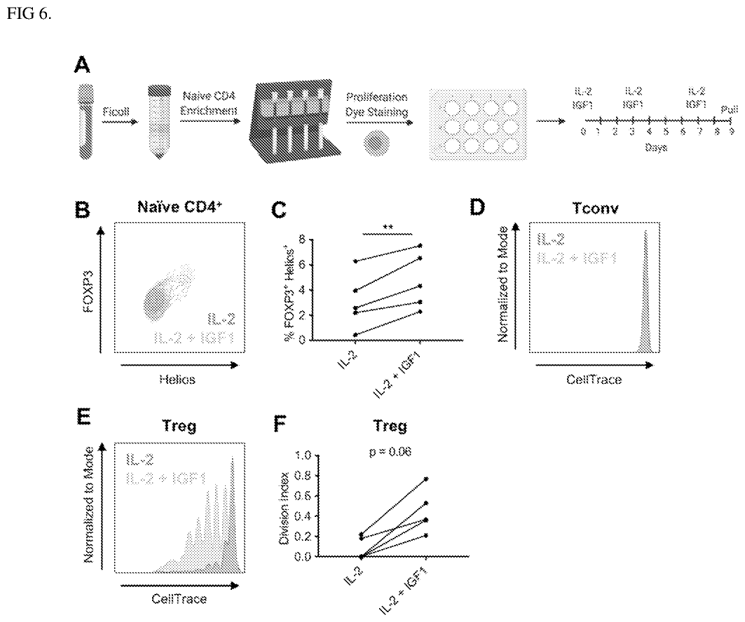

It was found using whole blood flow cytometry that naïve human Tregs express high levels of IGF1R, and the expression level is not impaired in subjects with T1D (FIG. 1-2). The expression of IGF1R decreases as the naïve Tregs mature into memory Tregs (FIG. 3). Human peripheral blood mononuclear cells were treated with IGF1, and Phosflow cytometry showed that naïve Tregs and Tconv responded to IGF1 treatment, while memory Tregs showed no response (FIG. 4-5). When naïve CD4⁺ T cells are cultured with IL-2 and IFG1 for several days, the amount of FOXP3+Helios+ Tregs increases while there are no changes in the amount of naïve FOXP3−Helios− Tconv (FIG. 6). Based on these findings, it has been determined that IGF1 can specifically augment naïve Treg proliferation in combination with IL-2 as a secondary stimulus.

The embodiment described within is a method for enabling lymphocyte proliferation and modification by combining signaling from the common gamma chain (γ_{c}) (or IL2RG, CD132) with the IGF1 growth factor. The specific embodiment described within is a method for targeted naïve Treg proliferation by treatment with a combination of IGF1 and IL-2. The Tregs maintain a naïve phenotype while undergoing cell division and after passaging. In some embodiments, the population of naïve Treg cells may provide therapeutic benefits in treating autoimmune diseases. The disclosure also features expanding naïve Treg populations after modification by gene editing and lentiviral transduction. This may be achieved using gene therapy vectors such as lentiviral gene delivery or via the CRISPR/Cas system to reduce or increase the expression of one or multiple genes. In some embodiments, the expansion of naïve Treg cells is performed in vivo by administering IGF1 and IL-2 to a subject with an autoimmune disease.

### Overview

Insulin-like growth factor-1 (IGF1) is an immunoregulatory hormone (15) that synergizes with IL-2 to promote naïve Treg proliferation, as IGF1R is known to be expressed at higher levels by naïve versus memory CD4⁺ T cells (18). It was recently shown that IGF1 levels are significantly reduced in pre-T1D (19), potentially contributing to the development of autoimmunity (16). In terms of bulk Treg enhancement, IGF1 has been shown to promote the in vitro proliferation of sorted human Tregs (17). Additionally, IGF1 treatment inhibited T1D development in the NOD and streptozotocin (STZ)-induced models, with the latter ascribed to increased Treg proliferation in peripheral blood, leading to elevated numbers of Tregs in the pancreas (17). Likewise, the development of murine experimental autoimmune encephalitis (EAE) (17) and allergic contact dermatitis (20) were inhibited by IGF1, with elevated numbers of Foxp3⁺ Tregs observed at the site of autoimmunity, as a direct consequence of Treg-specific IGF1:IGF1 receptor (IGF1R) signaling. These studies suggest that IGF1 may induce immunoregulation through preferential induction of Treg proliferation as compared to conventional CD4⁺ T cells (Tconv). Herein, IGF1R expression is characterized on naïve and memory Treg and Tconv subsets isolated from human subjects with and without T1D. Differential IGF1R signaling is demonstrated within CD4⁺ T cell subsets, allowing for the specific enhancement of naïve Treg proliferation by IGF1 in combination with low-dose IL-2.

The present disclosure is based on experiments quantifying IGF1R/CD221 expression on CD4⁺ T cell subsets from whole blood of human donors (FIG. 1A). Analyzing the total cohort, when comparing subsets within individual subjects, CD221 expression is significantly higher on naïve (CD45RA⁺CD197⁺) versus memory (CD45RA⁻) CD4⁺ T cells (FIG. 1B-C), in agreement with previous reports (18). Intriguingly, naïve Tregs (CD45RA⁺CD197⁺CD25^{hi}CD127^{lo/-}) show significantly higher CD221 expression than all other subsets assessed, including naïve Tconv (CD45RA⁺CD197⁺CD127⁺) (FIG. 1B-C), suggesting that naïve Tregs preferentially respond to IGF1.

The whole blood staining cohort is comprised of subjects with and without T1D in order to determine whether disease status impacted the degree of IGF1 signaling in CD4⁺ T cells. In contrast to known modulation of peripheral IGF1 levels in T1D (19), CD221 levels on naïve Tconv, memory Tconv, naïve Treg, and memory Treg are similar when comparing diabetes-free relatives to age- and sex-matched subjects with T1D (FIG. 2). These data suggest that IGF1R expression on CD4⁺ T cells is not impaired in T1D subjects.

IGF1R expression has previously been shown to decrease in human PBMCs with aging (21); however, immune subset-specific IGF1R expression has been poorly characterized. CD221 levels displayed a significant negative correlation with subject age is observed at draw in the naïve Tconv (R=−0.54, p=0.003) and naïve Treg (R=−0.58, p=0.001) compartments (FIG. 3A-B), and this association is weaker, but also apparent, in memory Tconv (R=−0.33, p=0.084) and memory Treg (R=−0.37, p=0.047) subsets (FIG. 3C-D). Collectively, the findings suggest that IGF1 may preferentially induce signaling in naïve Tregs, particularly in early life when T cell maturation remains active (22).

Differences in receptor expression suggest that IGF1 may preferentially induce IGF1R signaling in naïve as compared to memory CD4⁺ T cells; this question was yet to be formally experimentally tested. Therefore, whether IGF1 preferentially augmented phosphorylation of PI3K/Akt pathway targets, downstream of IGF1R, was measured in CD4⁺ T cell subsets in the context of T cell receptor (TCR) stimulation. Here, it is observed that pAkt (Ser473) was enhanced by IGF1 treatment to a significantly greater extent in naïve (CD45RA⁺) than in memory (CD45RA⁻) CD4⁺ T cells at 15 minutes (1.17-fold difference) and 30 minutes (1.17-fold difference, FIG. 3-4A-C). Likewise, downstream pS6 (Ser235/236) is upregulated by IGF1 to a greater extent in naïve than in memory CD4⁺ T cells at 15 minutes (1.23-fold difference) and 30 minutes (1.30-fold difference, FIG. 4D-E). Although it was observed that IGF1R expression was significantly higher on naïve Treg than naïve Tconv (FIG. 1), naïve FOXP3⁺Helios⁺ Treg show comparable IGF1R signaling induction to naïve FOXP3⁻Helios⁻ Tconv (FIGS). Together, these findings suggest that IGF1 may preferentially augment IGF1R signaling in activated naive CD4⁺ T cells. The observation that IGF1 augments TCR-mediated PI3K/Akt signaling in naïve CD4⁺ T cells implies that IGF1 could potentially synergize with other inducers of the PI3K/Akt pathway, such as cytokines. IL-2 has been shown to preferentially induce the homeostatic proliferation of naïve Tregs (23).

### Method for Expanding Naïve Treg Population

In this embodiment, the naïve CD4⁺ T cells are treated with IL-2 in combination with IGF1, to enhance naïve Treg proliferation (FIG. 6A). In certain aspects, naïve CD4⁺ T cells are isolated by either density gradient-centrifugation of whole blood followed by EasySep human naïve CD4⁺ T cell enrichment kit (19155, StemCell Technologies) or by RosetteSep human CD4⁺ T cell enrichment (StemCell Technologies) of whole blood followed by human CD45RO microbead depletion (Miltenyi Biotec).

In certain aspects naïve CD4⁺ T cells are plated at 1×10⁶ cells/mL in cRPMI with 20 IU/mL recombinant human IL-2 (Teceleukin) and 100 ng/mL recombinant human IGF1 (BioVision). In certain aspects, cytokine and/or growth factor are replenished on day 3 and day 7, assuming consumption, and intracellular flow cytometry is performed on day 9-11 to confirm the naïve phenotype of the Tregs.

In some embodiments, supplementation of low-dose IL-2 with IGF1 enhances the percentage of FOXP3⁺Helios⁺ Tregs within bulk naïve CD4⁺ T cell culture (1.54-fold change, FIG. 3-6 B-C). While low-dose IL-2+/−IGF1 does not induce the proliferation of naïve FOXP3⁻Helios⁻ Tconv (FIG. 6D), the addition of IGF1 promotes the expansion of naïve Tregs as compared to IL-2 alone (5.60-fold change, FIG. 6E-F). IGF1 enhances the capacity of low-dose IL-2 to specifically drive the homeostatic proliferation of naïve Treg.

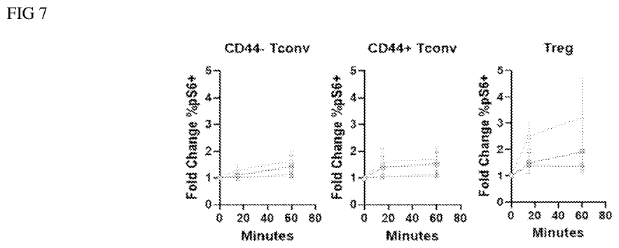

In certain embodiments IGF1 and LD IL-2 treatment stimulating Treg over Tconv is performed with murine cells. IGF1R and IL-2R signaling are assessed via phosflow of splenocytes from prediabetic NOD mice at 10-14 weeks of age. Cells are treated with 10 IU/mL human IL-2 (Teceleukin)±100 ng/mL human IGF1 (Biovision) for 15 or 60 minutes, followed by fixation, methanol permeabilization, and staining for pAkt (Ser473) and pS6 (Ser 235/236) for IGF1R signaling (24) and pSTAT5 (pY694) for IL-2R signaling (25, 26). The combinatorial IGF1 and LD IL-2 enhances pS6 expression beyond LD IL-2 alone, particularly in Tregs (FIG. 7).

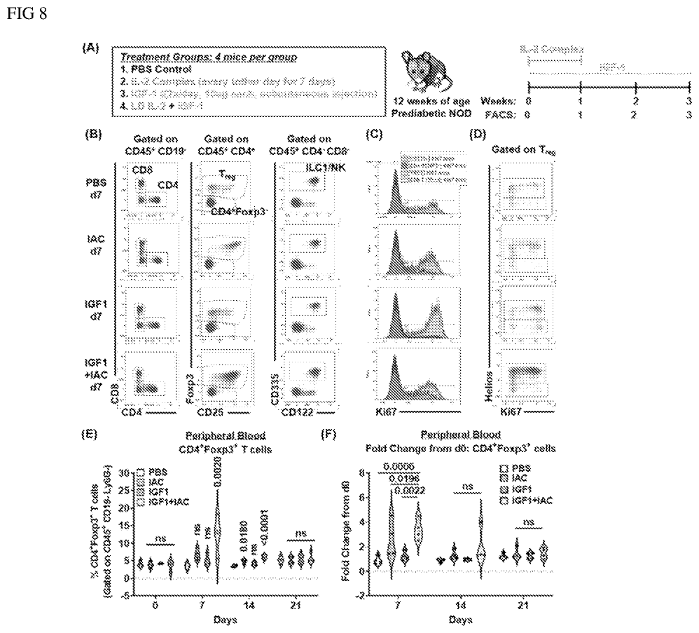

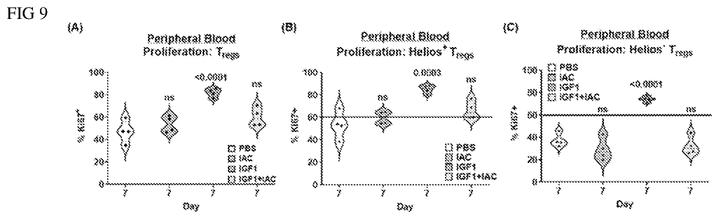

In certain embodiments prediabetic NOD mice are treated with IGF1 and/or IL-2 complex [IAC: anti-IL-2 (JES6.1)+rmIL-2] (FIG. 8A). The peripheral blood of treated NOD mice was examined for CD8, CD4+Foxp3−(Tconv), and CD4+Foxp3+ (Tregs) T-cells and Innate Lymphoid cells group 1 (ILC1, CD335+), which includes NK cells. As expected, IAC treatment for 1-week increases CD4+Foxp3+ Tregs (FIG. 8B, 8F) and expression of CD25 and Foxp3 and increases CD8 and NK cell proliferation (not shown). IAC does not increase the overall Treg proliferation (FIG. 9A), but rather results in the majority of proliferating Tregs to occur in the Helios+ population (59.61±2.82%) over the Helios− cells (29.10.61±5.03%) (FIG. 9B-C). Murine Helios+ Tregs are commonly considered tTregs, while Helios− Tregs are thought to be pTregs (27, 28), indicating that IL-2 strongly targets the tTreg population. In contrast, 1-week of IGF1 treatment results in significant Treg proliferation compared to PBS- and IAC-treated mice (FIG. 9A) and significantly increases proliferation in both the Helios+ and Helios− Treg populations (FIG. 9B-C), suggesting that IGF1 stimulates both tTreg and pTreg in NOD mice.

Importantly, the addition of IGF1 to the IAC resulted in a significant increase in the overall CD4+Foxp3+ Tregs (12.50±2.57%) compared to IGF1 or IAC alone (5.32±0.77% or 6.83±0.77%) or PBS control (3.54±0.41) 1-week after the start of treatment (FIG. 8E). This is also supported by a significant fold-increase in CD4+Foxp3+ Tregs from day 0 in the IGF1+IAC group (3.4±0.42) compared to IAC and IGF1 treatment alone (2.64±0.74 and 1.24±0.19) (FIG. 8F). In sum, it is observed that naïve Tregs express high levels of IGF1R, allowing for PI3K/Akt signaling that drives homeostatic proliferation in the context of IL-2. This represents a novel strategy toward rectifying the Treg:Tconv imbalance contributing to many autoimmune conditions.

### Gene Editing and Lentiviral Transduction

In other embodiments, naïve CD4+ T cell cultures are transduced with one or more vectors encoding a gene editing system engineered to reduce, prevent, increase, or otherwise alter expression of one or more genes. In certain aspects, modified naïve CD4+ T cell population is plated at 1×10⁶ cells/mL in cRPMI with 20 IU/mL recombinant human IL-2 (Teceleukin) and 100 ng/mL recombinant human IGF1 (BioVision). In certain aspects, cytokine and/or growth factor are replenished on day 3 and day 7, assuming consumption, and intracellular flow cytometry is performed on day 9-11. In this embodiment, supplementation of low-dose IL-2 with IGF1 enhances the percentage of gene modified FOXP3⁺Helios⁺ Tregs within the gene modified naïve CD4⁺ T cell culture. In one embodiment, the vectors are lentiviral encoding a de novo T-cell receptor (TCR) or Chimeric antigen receptor (CAR) T-cell for use in immunotherapy. In one embodiment, the gene editing system includes a nuclease for facilitating stable, site-specific recombination in a recipient host. Exemplary nuclease in accordance with this embodiment includes a Clustered Regularly Interspaced Short Palindromic Repeats (CRISPR)-associated (Cas), a protein a zinc finger nuclease (ZFN), a Transcription Activator-Like Effector Nuclease (TALEN), or a meganuclease.

In some embodiments, a CRISPR/Cas system is utilized to induce a single or a double strand break in the target cell's genome. CRISPR (Clustered Regularly Interspaced Short Palindromic Repeats) is an acronym for DNA loci that contain multiple, short, direct repetitions of base sequences. By transfecting a cell with the required elements including a Cas gene and specifically designed CRISPRs, the organism's genome can be cut and modified at any desired location. Methods of preparing compositions for use in genome editing using the CRISPR/Cas systems are described in U.S. Pat. No. 8,697,359 and US 2014-0068797.

In general, the “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. One or more tracr mate sequences operably linked to a guide sequence (e.g., direct repeat-spacer-direct repeat) can also be referred to as pre-crRNA (pre-CRISPR RNA) before processing or crRNA after processing by a nuclease.

In some embodiments, a tracrRNA and crRNA are linked and form a chimeric crRNA-tracrRNA hybrid where a mature crRNA is fused to a partial tracrRNA via a synthetic stem loop to mimic the natural crRNA: tracrRNA duplex as described in Cong et al. and Jinek et al. above, A single fused crRNA-tracrRNA construct can also be referred to as a guide RNA or gRNA (or single-guide RNA (sgRNA)). Within an sgRNA, the crRNA portion can be identified as the “target sequence” and the tracrRNA is often referred to as the “scaffold”.

There are many resources available for helping practitioners determine suitable target sites once a desired DNA target sequence is identified. For example, numerous public resources, including a bioinformatically generated list of about 190,000 potential sgRNAs, targeting more than 40% of human exons, are available to aid practitioners in selecting target sites and designing the associate sgRNA to affect a nick or double strand break at the site. See also, crispr.u-psud.fr/, a tool designed to help scientists find CRISPR targeting sites in a wide range of species and generate the appropriate crRNA sequences.

In some embodiments, one or more vectors driving expression of one or more elements of a CRISPR system are introduced into a target cell such that expression of the elements of the CRISPR system direct formation of a CRISPR complex at one or more target sites. While the specifics can be varied in different engineered CRISPR systems, the overall methodology is similar. In certain preferred embodiments, the CRISPR system is used to target a DNA sequence by inserting a short DNA fragment containing the target sequence into a guide RNA expression plasmid. A suitable sgRNA expression plasmid may contain the target sequence (about 20 nucleotides), a form of the tracrRNA sequence (the scaffold) as well as a suitable promoter and necessary elements for proper processing in eukaryotic cells. Such vectors are commercially available {see, e.g., Addgene, a plasmid repository in Cambridge, MA). Many of the systems rely on custom, complementary oligos that are annealed to form a double stranded DNA and then cloned into the sgRNA expression plasmid. Co-expression of the sgRNA and the appropriate Cas enzyme from the same or separate plasmids in transfected cells results in a single or double strand break (depending of the activity of the Cas enzyme) at the desired target site.

In some embodiments of the methods described herein, ZFNs may be used to inhibit the expression of one or more genes. “Zinc finger nucleases” or “ZFNs” are a fusion between the cleavage domain of Fokl and a DNA recognition domain containing 3 or more zinc finger motifs. The heterodimerization at a particular position in the DNA of two individual ZFNs in precise orientation and spacing leads to a double-strand break in the DNA. In some cases, ZFNs fuse a cleavage domain to the C-terminus of each zinc finger domain. In order to allow the two cleavage domains to dimerize and cleave DNA, the two individual ZFNs bind opposite strands of DNA with their C-termini at a certain distance apart. In some cases, linker sequences between the zinc finger domain and the cleavage domain requires the 5′ edge of each binding site to be separated by about 5-7 bp.

In some embodiments, TALENs can be used to generate gene modifications by creating a double-strand break in a target DNA sequence, which in turn, undergoes NHEJ or HDR. In some cases, a single-stranded donor DNA repair template is provided to promote HDR. TALENs” or “TAL-effector nucleases” are engineered transcription activator-like effector nucleases that contain a central domain of DNA-binding tandem repeats, a nuclear localization signal, and a C-terminal transcriptional activation domain.

In some embodiments of the methods described herein, meganucleases may be used to inhibit the expression of one or more genes. “Meganucleases” are rare-cutting endonucleases or homing endonucleases that can be highly specific, recognizing DNA target sites ranging from at least 12 base pairs in length, e.g., from 12 to 40 base pairs or 12 to 60 base pairs in length. Meganucleases can be modular DNA-binding nucleases such as any fusion protein comprising at least one catalytic domain of an endonuclease and at least one DNA binding domain or protein specifying a nucleic acid target sequence. The DNA-binding domain can contain at least one motif that recognizes single- or double-stranded DNA. The meganuclease can be monomeric or dimeric.

In some embodiments, naïve CD4⁺ T cell cultures are transfected with one or more lentiviral vectors encoding a de novo T-cell receptor (TCR) or Chimeric antigen receptor (CAR) T-cell for use in immunotherapy. The delivery of a gene(s) or other polynucleotide sequences using a retroviral or lentiviral vector by means of viral infection rather than by transfection is referred to as “transduction.” In one embodiment, retroviral vectors are transduced into a cell through infection and provirus integration. In certain embodiments, a cell, e.g., a target cell, is “transduced” if it comprises a gene or other polynucleotide sequence delivered to the cell by infection using a viral or retroviral vector. In certain embodiments, a transduced cell comprises one or more genes or other polynucleotide sequences delivered by a retroviral or lentiviral vector in its cellular genome.

The naive CD4⁺ T cell are isolated from a subject and undergo lentiviral transduction to express a CAR or a de novo TCR targeting a tumor associated antigen. First generation CARs are composed of an extracellular binding domain, a hinge region, a transmembrane domain, and one or more intracellular signaling domains. Currently, Fourth generation CARs (also known as TRUCKs or armored CARs) further add factors that enhance T cell expansion, persistence, and anti-tumoral activity. This can include cytokines, such is IL-2, IL-5, IL-12 and co-stimulatory ligands. TCRs use heterodimers consisting of alpha and beta peptide chains to recognize polypeptide fragments presented by MHC molecules. The modified naïve CD4+ T cell population is cultured with IL-2 and IFG1 to enhance the percentage of gene modified FOXP3⁺Helios⁺ Tregs within the gene modified naïve CD4⁺ T cell culture. The TCR or CAR naïve Treg is administered to a subject for immunotherapy.

### Applications and Treatments

The disclosure further provides a method of administering naive Treg proliferated from CD4+ naïve T cells isolated from a subject; naive Treg proliferated from CD4+ naïve T cells isolated from a donor subject; genetically modified naïve Tregs proliferated from a subject or donor subject; or a mixture thereof and a pharmaceutically acceptable carrier and/or excipient. In some embodiments, the pharmaceutical composition comprises IGF1, a common gamma chain, and a pharmaceutically acceptable carrier. In a specific embodiment, the pharmaceutical composition comprises IGF1, IL-2, and a pharmaceutically acceptable carrier. Optionally IL-2 maybe co-administered with an antilL-2 antibody to extend the half-life of IL-2.

Pharmaceutical compositions, as disclosed herein, can be formulated in accordance with standard pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York) known by a person skilled in the art. Pharmaceutical composition according to the disclosure may also be formulated to release active agents (e.g., Treg as disclosed herein alone or in combination with another agent) substantially immediately upon administration or at any predetermined time or time period after administration.

Compositions for parenteral administration are generally physiologically compatible sterile solutions or suspensions that can optionally be prepared immediately before use from solid or lyophilized form. Adjuvants, local anesthetics, preservatives and/or buffering agents can be added to the vehicle and a surfactant or wetting agent can be included in the composition to facilitate uniform distribution of the active ingredient.

The composition can be formulated into conventional dosage forms, such as liquid preparations, syrups, and concentrated drops. Non-toxic solid carriers or diluents may be used, which include, for example, pharmaceutical grades of mannitol, lactose, starch, magnesium stearate, sodium saccharine, talcum, cellulose, glucose, sucrose, magnesium, carbonate.

The pharmaceutical composition of the disclosure may be in a variety of forms. These include, for example, solid, semi-solid, and liquid dosage forms, such as lyophilized preparations, liquids solutions or suspensions, injectable and infusible solutions, etc. The preferred form depends on the intended mode of administration and therapeutic application.

The amount of the therapeutic or pharmaceutical composition of the disclosure that is effective in the prevention and/or treatment of autoimmune diseases can be determined by a person skilled in the art having the benefit of the current disclosure through standard clinical techniques. The precise dose to be employed in the formulation will also depend on several aspects, including but not limited to, the route of administration, the subject being treated, and the severity of the disease, and should be decided according to the judgment of the practitioner and each patient's circumstances. In one embodiment, effective doses can be extrapolated from dose-response curves derived from in vitro or animal model test systems.

In many instances, it will be desirable to have multiple administrations of the composition, e.g., 2 administrations provided daily, or 1 administration provided on alternating days. A normal regimen will often occur in one-to-three-week intervals. Periodic administration at intervals of 1-5 years, may be desirable to maintain levels of a naïve Treg population.

The disclosure provides methods of delivery and transplantation of the specially culture Tregs to ameliorate the effects of autoimmune disease in a subject. Transplantation of naïve Tregs into a subject in need has the potential to prevent or halt the progression of autoimmune disease. The disclosure provides methods, for generating naïve Tregs adapted to maintain a naïve phenotype in mammalian tissue when introduced thereto. The use of the Tregns in the treatment of autoimmune diseases can be demonstrated by the use of established animal models known in the art.

Tregs of the disclosure can be administered to an animal with autoimmune diseases obtained in any manner, including those obtained as a result of age or animal models created by man using recombinant genetic techniques, such as transgenic and “gene knockout” animals.

Recipients of the Tregs of the disclosure can be immunosuppressed, either through the use of immuno suppressive drugs such as cyclosporin, or through local immunosuppression strategies employing locally applied immunosuppressants, but such immunosuppression need not necessarily be a prerequisite in certain immunoprivileged tissues.

Tregs of the disclosure can be prepared from the recipient's own tissue. In such instances, the Tregs can be generated from dissociated or isolated tissue and proliferated in vitro using the methods described herein. Upon suitable expansion of cell numbers, Tregs of the disclosure can be harvested, treated according to the methods of the disclosure and readied for administration into the recipient.

### EXAMPLES

### Example 1. Methods

Murine IGF1R Staining. Spleen and thymus of six-week-old NOD.Foxp3-GFP/cre (Stock No: 008694, Jackson Laboratory) were processed using frosted glass slides and passed through a 40-micron filter to create a single cell suspension. Red blood cells (RBC) were lysed with ammonium-chloride-potassium buffer prior to staining for flow cytometry analysis. Samples were stained with Fixable Live/Dead Near IR (Invitrogen), and Fc receptors were blocked with anti-CD16/32 (Clone 2.4G2, BD Biosciences). Antibodies used included CD3e-Brilliant Violet (BV) 605 (145-2C11, BioLegend), CD4-PerCP/Cy5.5 (RM4-5, Thermo Fisher Scientific), CD8a-BV711 (53-6.7, BioLegend), CD44-PE-Cy7 (IM7, BioLegend), CD62L-APC (MEL-14, BioLegend), and CD221-PE (3B7, Santa Cruz Biotechnology). Data were acquired on an LSRFortessa (BD Biosciences) and analyzed with FlowJo (v10.6.1; Tree Star).

Murine IGF1R Signaling. Single cell suspensions of splenocytes were generated from 11-17-week-old pre-diabetic NOD mice (Stock No: 001976, Jackson Laboratory), followed by RBC lysis. Cells were resuspended at 1e6/mL in complete RPMI (cRPMI) 1640 without L-glutamine (Corning) supplemented with 2 mM GlutaMAX (Gibco), 100 IU and 100 μg/mL each of penicillin-streptomycin solution (Corning), 1 mM sodium pyruvate (Corning), 0.1 mM non-essential amino acids (Gibco), 10 mM HEPES (Gibco), 10% fetal bovine serum (GenClone), and 0.0004% 2-mercaptoethanol (Sigma-Aldrich) and treated with 10 IU/mL recombinant human (rh)-IL-2 (Teceleukin) and/or 100 ng/mL rhIGF1 (BioVision) for 15 or 60 minutes at 37° C. Samples were fixed with an equal volume of Cytofix fixation buffer (BD Biosciences) for 10 minutes at 37° C. Live/Dead fixable near-IR dead cell stain kit (Invitrogen) was applied according to manufacturer's instructions for dead cell exclusion. Cells were washed once with stain buffer, permeabilized with Phosflow perm buffer III (BD Biosciences) for 30 minutes on ice, washed twice with stain buffer, and then incubated with anti-CD16/32 (Clone 2.4G2, BD Biosciences) for 5 minutes at 23° C. Samples were stained with the following fluorescently-labeled anti-mouse antibodies at 23° C. for 45 minutes: CD3-PerCP-Cy5.5 (17A2), CD4-BV711 (RM4-5), CD44-PE-Cy7 (IM7), Helios-Pacific Blue (22F6) (BioLegend), Foxp3-AlexaFluor (AF)-AF488 (FJK-16s, eBioscience), pS6 Ser235/236-AF647 (D57.2.2E, Cell Signaling Technology), and pSTAT5 Tyr694-PE (47/STATS, BD Biosciences). Samples were washed once with stain buffer prior to data acquisition on an LSRFortessa (BD Biosciences) and analysis with FlowJo software (v10.6.1; Tree Star).

Human Subject Enrollment. Relatives of T1D subjects were age- and sex-matched to T1D patients, who were recruited from the 2018 Children with Diabetes-sponsored Friends for Life (FFL) Orlando Conference. Detailed deidentified subject demographic information is presented in Table 3-1 for whole blood staining study performed with FFL samples. Additional subjects were recruited from general population or clinics at the University of Florida (UF; Gainesville, FL); Nemours Children's Hospital (Orlando, FL); and Emory University (Atlanta, GA) for the remainder of experiments performed (Table 3-2, Table 3-3). Peripheral blood samples were collected from non-fasted subjects by venipuncture in heparin-coated vacutainer tubes (BD Biosciences).

Human Whole Blood Flow Cytometry. 200 μL of whole blood was stained with fluorescently-labeled anti-human antibodies for thirty minutes at 23° C. in the dark. To determine IGF1R expression, the following antibodies were used: CD25-AF488 (BC96), CD127-BV421 (A019D5), CD197-AF647 (G043H7), CD45RA-PerCP-Cy5.5 (HI100), CD3-APC-Fire750 (SK7), CD221-PE (1H7) (BioLegend), CD4-PE-Cy7 (RPA-T4, eBioscience). Red blood cells were lysed for five minutes at 23° C. with eBioscience 1-step Fix/Lyse Solution (ThermoFisher), followed by three washes with staining buffer (PBS+2% FBS+0.05% NaN₃). Data were acquired on an LSRFortessa (BD Biosciences) and analyzed with FlowJo software (v10.6.1; Tree Star).

Human IGF1R Signaling. Whole blood was processed to peripheral blood mononuclear cells (PBMCs) via density gradient centrifugation. PBMCs were incubated overnight at 1×10⁶ cells/mL with 2 μg/mL of ultra-low endotoxin, azide free-purified anti-human CD3 (OKT3, BioLegend) and 1 μg/mL of no azide/low endotoxin-purified anti-human CD28 (CD28.2, BD Biosciences) in cRPMI. The following day, samples were stimulated with 100 ng/mL recombinant human (rh) IGF1 (BioVision) for 15 or 30 minutes at 37° C. and then immediately fixed with an equal volume of Cytofix fixation buffer (BD Biosciences) for 10 minutes at 37° C. Live/Dead fixable near-IR dead cell stain kit (Invitrogen) was applied according to manufacturer's instructions for dead cell exclusion. Cells were washed once with stain buffer, permeabilized with Phosflow perm buffer III (BD Biosciences) for 30 minutes on ice, washed twice with stain buffer, and then incubated with human TruStain FcX (BioLegend) for 5 minutes at 23° C. Samples were stained with the following fluorescently-labeled anti-human antibodies at 23° C. for 45 minutes: CD3-PerCP-Cy5.5 (UCHT1), CD45RA-BV711 (HI100), FOXP3-AF488 (206D), FOXP3-AF488 (259D), Helios-Pacific Blue (22F6) (BioLegend), CD4-PE-Cy7 (RPA-T4, eBioscience), pS6 Ser235/236-AF647 (D57.2.2E), and pAkt Ser473-PE (D9E) (Cell Signaling Technology). Samples were washed once with stain buffer prior to data acquisition on an LSRFortessa (BD Biosciences) and analysis with FlowJo software (v10.6.1; Tree Star).

In vitro Homeostatic Proliferation. Naïve CD4⁺ T cells were isolated by either density gradient-centrifugation of whole blood followed by EasySep human naïve CD4⁺ T cell enrichment kit (19155, StemCell Technologies) or by RosetteSep human CD4⁺ T cell enrichment (StemCell Technologies) of whole blood followed by human CD45RO microbead depletion (Miltenyi Biotec). Enriched cells were stained with Cell Proliferation Dye eFluor670 (Thermo Fisher) according to manufacturer's instructions. Naïve CD4⁺ T cells were plated at 1×10⁶ cells/mL in cRPMI with 20 IU/mL recombinant human (rh) IL-2 (Teceleukin) and 100 ng/mL rhIGF1 (BioVision). Cytokine and/or growth factor were replenished on day 3 and day 7, assuming consumption, and intracellular flow cytometry was performed on day 9-11. Live/Dead fixable near-IR dead cell stain kit (Invitrogen) was applied according to manufacturer's instructions for dead cell exclusion. Cells were washed once with stain buffer and incubated with human TruStain FcX (BioLegend) for 5 minutes on ice. Samples were stained with the following fluorescently-labeled anti-human antibodies on ice for 30 minutes: CD4-PerCP-Cy5.5 (RPA-T4), CD45RA-BV711 (HI100), and CD221-PE (1H7) (BioLegend) prior to fixation and permeabilization with FOXP3 transcription factor staining buffer set according to manufacturer's protocol (eBioscience). Intracellular staining was performed with the following fluorescently-labeled anti-human antibodies for 30 minutes at 23° C.: FOXP3-AF488 (206D), FOXP3-AF488 (259D), and Helios-Pacific Blue (22F6) (BioLegend). Data were acquired on an LSRFortessa (BD Biosciences) and analyzed with FlowJo software (v10.6.1; Tree Star).

Murine IGF1+Low-Dose IL-2 Treatment. Prediabetic 12-week old female NOD mice (Stock No: 001976, Jackson Laboratory) were treated with IL-2 antibody complex [IAC: 5 μg anti-IL-2 (JES6.1)+1 μg recombinant mouse IL-2] injected intraperitoneally every other day for a week (33), 10 μg rhIGF1 injected subcutaneously twice a day for three weeks (32), IAC+IGF1 in combination, or sham control (PBS). Peripheral blood was collected before treatment and on a weekly basis during the treatment regimen for flow cytometric analysis. RBC were lysed prior to staining with Live/Dead fixable near-IR dead cell stain kit (Invitrogen). Cells were stained with the following antibodies: CD19-APC-Cy7 (1D3, BD Biosciences), Ly6G-APC-Cy7 (1A8), CD122-Biotin (TM-β1), CD45-BV510 (30-F11), CD5-BV650 (53-7.3), CD4-PE-Cy7 (RM4-5), CD8-AF700 (53-6.7), CD25-PE-CF594 (PC61), CD335-PE (29A1.4), cKIT (CD117)-Brilliant Blue (BB)-700 (2B8), CD44-BV605 (1M7), prior to a wash with stain buffer and application of a Streptavidin-BV711 conjugate. Samples were fixed and permeabilized with Foxp3 transcription factor staining buffer set according to manufacturer's protocol (eBioscience) before staining with anti-mouse Helios-APC (22F6), Foxp3-eFluor 450 (FJK-16s), Eomes-AF488 (Dan11ma), and Ki67-BV786 (B56).

Human T cell Transduction. PBMC were isolated from whole blood by density gradient centrifugation and naïve Treg fluorescence-activated cell sorted (FACS) as CD45RA⁺CD197⁺CD25^{hi}CD127^{lo/-} using a FACSAria III (BD Biosciences). Naïve Treg were stained with Cell Proliferation Dye eFluor670 (eBioscience) and cultured at 1e6/mL with 20 IU/mL rhlL-2 (Teceleukin) and/or 100 ng/mL rhIGF1 (BioVision) for 7 days prior to transduction. Cells were transduced using 8 μg/mL protamine sulfate (Sigma-Aldrich) and 3 TU/cell lentiviral vector containing R164, a T cell receptor (TCR) recognizing T1D-relevant epitope glutamic acid decarboxylase 65 (GAD65) 555-567 in the context of HLA-DRB1*04:01 (53). Lentiviral vector composition (33) and production (34) were as previously described. Cells were spinoculated by centrifugation at 1000×g for 30 min at 32° C. (34) and IL-2 and/or IGF1 added assuming consumption on days 3, 7, 10, and 14. Transduction efficiency and maintenance of naivety were evaluated on day 14 via enhanced green fluorescence protein (eGFP) reporter and CD45RA⁺CD197⁺ co-expression, respectively. Proliferation was quantified by calculation of proliferation index from Cell Proliferation Dye eFluor670 signal.

Statistics. Analyses were performed using GraphPad Prism software version 7.0. Data are presented as mean±standard deviation (SD) and all tests were two-sided unless otherwise specified. Murine IGF1R signaling was compared between treatment groups and timepoints using repeated measures two-way ANOVA with Tukey's multiple comparisons test. One-way ANOVA with Dunnett's or Tukey's multiple comparisons tests were used to assess outcomes of in vivo IL-2+IGF1 treatment when comparing all treatment groups to each other or only to PBS control, respectively. Friedman test with Dunn's multiple comparisons test or Mann-Whitney U test were used to compare IGF1R expression on CD4⁺ T cell subsets within human subjects or between T1D subjects and relatives, respectively. Associations between IGF1R levels and subject age were assessed via Spearman correlation. Human IGF1R signaling and homeostatic proliferation were compared between cell subsets or experimental conditions via Wilcoxon test or paired t-test, depending upon if the data were normally distributed by Shapiro-Wilk test. P-values<0.05 were considered significant.

Study approval. All procedures with human samples were approved by Institutional Review Boards at each institution and conducted in accordance with the Declaration of Helsinki. Written informed consent was obtained from participants (or their legal guardian in the case of minors) prior to enrollment. NOD/ShiLtJ mice were bred and housed in specific pathogen-free facilities at the University of Florida (UF) or University of Miami (UM), with food and water available ad libitum. All murine studies were conducted in accordance with protocols approved by the UF or UM Institutional Animal Care and Use Committee (IACUC) and in accordance with the National Institutes of Health Guide for Care and Use of Animals.

### Example 2. IGF1R Expression During T Cell Development and Activation in NOD Mice

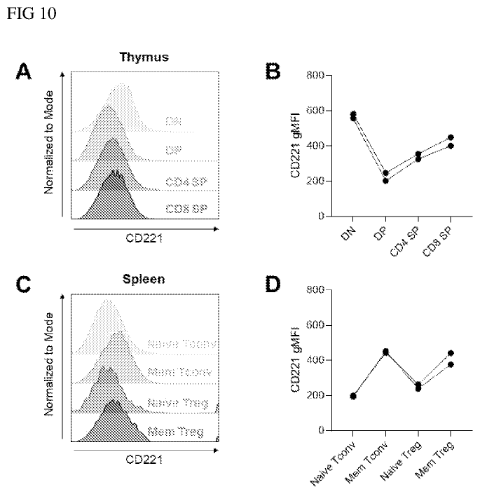

To elucidate the means by which IGF1 promotes T cell regulation, IGF1R expression was measured by flow cytometry on NOD. Foxp3-GFP thymocyte and splenocyte populations through the stages of central development and peripheral activation, respectively. IGF1R expression was highest in CD4⁻CD8⁻ double negative (DN) thymocytes as compared to later developmental stages (FIG. 10A-B), in agreement with a previous study showing that human DN thymocytes express approximately three times more surface IGF1R protein than CD4⁺CD⁺ double positive (DP) thymocytes (29). Interestingly, IGF1R expression rebounded in CD4⁺CD8⁻ and CD4⁻CD8⁺ single positive (SP) thymocytes (FIG. 10A-B), in contrast to observations in human thymocytes (29). Moving to the periphery, splenic CD44^{hi}CD62L⁻ memory CD4⁺ T cells showed significantly higher levels of IGF1R than CD44^{lo}CD62L⁺ naïve CD4⁺ T cells (FIG. 10C-D), as previously reported in Balb/c mice (30). While this expression pattern was replicated upon comparing naïve and memory CD4⁺Foxp3⁺ Treg, a modest enhancement of IGF1R expression was observed in naïve Treg versus naïve CD4⁺Foxp3⁻ Tconv (FIG. 10C-D), suggesting that differential IGF1R levels may partially account for IGF1-mediated T cell regulation.

### Example 3. IGF1 and IL-2 Synergize In Vitro to Preferentially Induce IGF1R Signaling in NOD Tregs

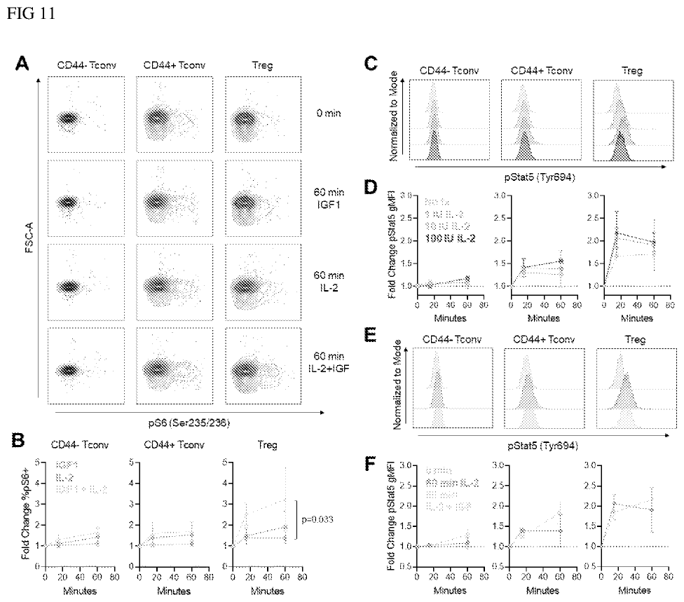

IGF1R signaling was assessed in splenocytes from NOD mice via Phosflow staining to determine whether IGF1R protein expression directly correlated with magnitude of IGF1R signaling. Modest IGF1-mediated induction of pS6 Ser235/236, a measure of PI3K/Akt signaling downstream of IGF1R, was observed in CD4⁺Foxp3⁺Helios⁺ Tregs (1.38-fold increase at 15- and 60-minutes post-treatment, FIG. 11A-B). Thus, it was hypothesized that IGF1 could potentially synergize with other inducers of the PI3K/Akt pathway, such as cytokines, to augment Treg IGF1R signaling. In particular, IL-2 has previously been shown to preferentially induce the proliferation of Tregs (31). Therefore, murine splenocytes were treated with low-dose IL-2 in combination with IGF1, in order to test whether IGF1 could further enhance Treg PI3K/Akt signaling. Indeed, the combination enhanced Treg-specific pS6 expression in a synergistic manner (2.50- and 3.22-fold increase at 15 and 60 minutes, respectively) beyond that of either agent alone (IGF1: 1.38-fold increase at 15- and 60-minutes, IL-2: 1.49- and 1.91-fold increase at 15- and 60-minutes post-treatment, respectively, FIG. 11A-B). Importantly, the synergy was specific to the Treg compartment, as the combination treatment did not significantly increase pS6 expression in naïve or memory Tconv (FIG. 11A-B). In contrast to the PI3K/Akt pathway, IL-2R signaling, as demonstrated by pSTAT5 Tyr694 induction by IL-2 (FIG. 11C-D), was unaffected by the addition of IGF1 (FIG. 11E-F). Together, these data imply that IGF1 and IL-2 are capable of synergizing to promote Treg-specific IGF1R signaling and thereby, may promote downstream effects of PI3K/Akt signaling such as cellular proliferation and survival.

### Example 4. IGF1 and IL-2 Synergize In Vivo to Specifically Promote Treg Proliferation

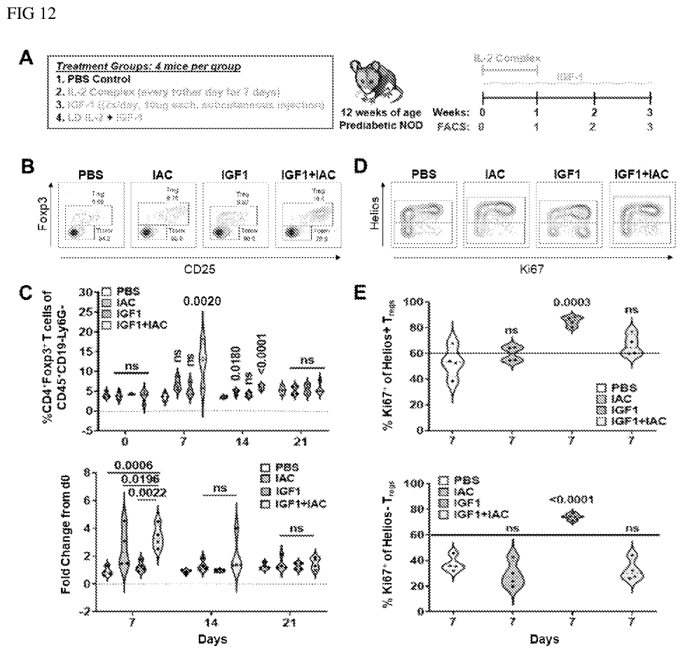

To assess whether the in vitro observation of IGF1 and IL-2 treatment supporting Treg-specific IGF1R signaling would translate to in vivo immune regulation, pre-diabetic NOD mice were treated with 10 μg rhIGF1 twice daily for three weeks (32) and/or IL-2 antibody complex [IAC: 5 μg anti-IL-2 (JES6.1)+1 μg recombinant mouse IL-2 (rmIL-2)], a method commonly used to extend the half-life of low-dose IL-2, every other day for one week (33) (FIG. 12A). Populations known to proliferate in response to IL-2 treatment were examined including CD8⁺, CD4⁺Foxp3⁻ (Tconv), and CD4⁺Foxp3⁺ (Tregs) T cells as well as Innate Lymphoid cells group 1 (ILC1, CD335⁺), which includes NK cells, in the peripheral blood of treated mice. As expected, IAC treatment for one week increased the CD4⁺Foxp3⁺ Treg percentage (FIG. 12B-C) with some off-target CD8⁺ T cell and NK cell proliferation. While IAC did not significantly increase overall Treg proliferation, the majority of proliferating Tregs occurred in the Helios⁺ thymically-derived (tTregs, 59.61±2.82%) over the Helios⁻ peripherally-induced (pTreg) population (29.10.61±5.03%) (FIG. 12D-E) (34, 35). In contrast, one week of IGF1 treatment resulted in significantly increased Treg proliferation compared to PBS- and IAC-treated mice, with proliferation observed in both the Helios⁺ and Helios⁻ Treg populations (FIG. 12D-E), suggesting that IGF1 can stimulate both tTreg and pTreg in vivo. Importantly, the combination of IGF1 plus IAC resulted in a significant increase in overall CD4⁺Foxp3⁺ Treg percentage (12.50±2.57%) compared to IGF1 or IAC alone (5.32±0.77% or 6.83±0.77%, respectively) or PBS control (3.54±0.41) at one week after the start of treatment (FIG. 12B-C) without increasing off-target CD8⁺ T cell or ILC1/NK cell proliferation beyond IAC alone. This resulted in a significant fold-increase in CD4⁺Foxp3⁺ Tregs from baseline in the IGF1+IAC group (3.4±0.42) compared to IAC and IGF1 treatment alone (2.64±0.74 and 1.24±0.19) (FIG. 12B-C). While percentage of CD4⁺Foxp3⁺ Tregs remained increased in the IGF1+IAC group at two weeks post-treatment as compared to PBS control, the levels were already beginning to contract to those observed pre-treatment and were no longer statistically different from other treatment groups by three weeks post-treatment (FIG. 12B-C). These findings suggest that IGF1 and low-dose IL-2 synergize to transiently promote Treg-specific proliferation in vivo and beg the question of whether the combination treatment would impact human immune cells similarly.

### Example 5. Naïve Human Tregs Express High Levels of IGF1R

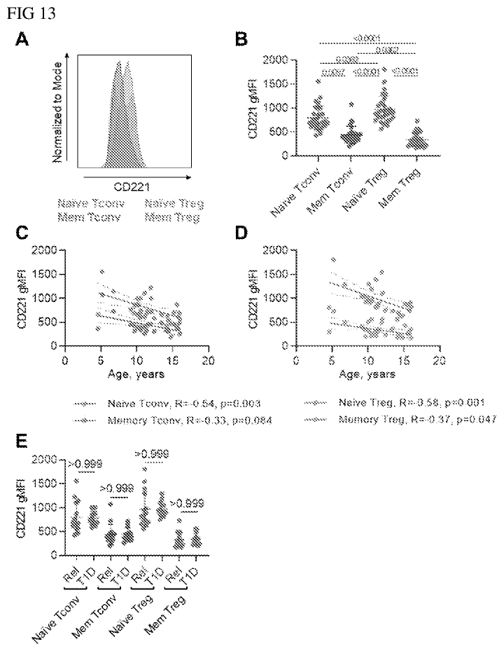

To understand if differential IGF1R levels observed on T cell subsets from the T1D-prone NOD mouse may translate to human subjects, IGF1R (CD221) expression was quantified by flow cytometry on CD4⁺ T cell subsets from fresh whole blood of children aged 4-16 years with and without T1D (n=14 and n=15, respectively; Table 1). Analyzing the total cohort together, IGF1R expression was significantly higher on naïve (CD45RA⁺CD197⁺) versus memory (CD45RA⁻) CD4⁺ T cells (FIG. 13A-B), in agreement with previous reports (34). Intriguingly, naïve Tregs (CD45RA⁺CD197⁺CD25^{hi}CD127^{lo/-}) showed significantly higher IGF1R expression than all other subsets assessed, including naïve Tconv (CD45RA⁺CD197⁺CD127⁺) (FIG. 13A-B), suggesting that naïve Tregs may preferentially respond to IGF1.

IGF1R mRNA expression has previously been shown to decrease in human peripheral blood mononuclear cells (PBMCs) with aging in adult subjects (36); however, immune subset-specific IGF1R expression has been poorly characterized. It was observed that IGF1R levels displayed a significant negative correlation with subject age in a pediatric cohort in the naïve Tconv (R=−0.54, p=0.003, FIG. 13C) and naïve Treg (R=−0.58, p=0.001, FIG. 13D) compartments, and this association was weaker, but also apparent, in memory Tconv (R=−0.33, p=0.084, FIG. 13C) and memory Treg (R=−0.37, p=0.047, FIG. 13D) subsets. Collectively, the findings suggest that IGF1 may preferentially induce signaling in naïve Tregs, particularly in early life when T cell maturation remains active (37).

IGF1R expression on T cell subsets from subjects with and without T1D (Table 1) was compared in order to determine whether disease status might impact the degree of IGF1 signaling in CD4⁺ T cells. In contrast to known modulation of peripheral IGF1 levels in T1D (19), IGF1R levels on naïve Tconv, memory Tconv, naïve Treg, and memory Treg were similar when comparing diabetes-free relatives to age- and sex-matched subjects with T1D (FIG. 13E). These data suggest that IGF1R expression on CD4⁺ T cells is not impaired in T1D subjects.

TABLE 1
Demographic information for relatives and T1D subjects
enrolled in whole blood IGF1R expression study.

| Cohort | Relatives | T1D |
|---|---|---|
| Total Subjects, n | 15 | 14 |
| Sex, n (%) | | |
| Male | 7 (47) | 7 (50) |
| Female | 8 (53) | 7 (50) |
| Age (years) | 12.3 ± 3.2 | 10.9 ± 2.8 |
| Height (m)* | 1.5 ± 0.2 | 1.4 ± 0.2 |
| Weight (kg)* | 45.4 ± 17.5 | 39.1 ± 13.7 |
| BMI (kg/m²)* | 18.2 ± 3.7 | 18.8 ± 3.7 |
| Ethnicity, n (%) | | |
| Caucasian | 13 (87) | 7 (50) |
| Hispanic | 0 (0) | 3 (21) |
| African-Am | 0 (0) | 1 (7) |
| Asian/Pac-Isl | 1 (7) | 0 (0) |
| Other | 1 (7) | 3 (21) |
| Disease Duration (years) | N/A | 3.0 ± 2.3 |

*Provision of height and weight was voluntary; thus, these data are available for some, but not all study subjects.

### Example 6. IGF1 Preferentially Signals to Naïve Versus Memory CD4⁺ T Cells

Differences in receptor expression suggest that IGF1 may preferentially induce IGF1R signaling in naïve as compared to memory CD4⁺ T cells; although this question has yet to be formally experimentally tested. Therefore, it was measured whether IGF1 preferentially augmented phosphorylation of PI3K/Akt pathway targets, downstream of IGF1R, in human CD4⁺ T cell subsets in the context of T cell receptor (TCR) stimulation (Table 2). Here, it was observed that pAkt (Ser473) was enhanced by IGF1 treatment to a significantly greater extent in naïve (CD45RA⁺) than in memory (CD45RA⁻) CD4⁺ T cells at 15 minutes (1.17-fold difference) and 30 minutes (1.17-fold difference). Likewise, downstream pS6 (Ser235/236) was upregulated by IGF1 to a greater extent in naïve than in memory CD4⁺ T cells at 15 minutes (1.23-fold difference) and 30 minutes (1.30-fold difference). Although it was observed that IGF1R expression was significantly higher on naïve Treg than naïve Tconv (FIG. 13), naive FOXP3⁺Helios⁺ Treg showed comparable IGF1R signaling induction to naïve FOXP3⁻Helios⁻ Tconv. Together, these findings suggest that IGF1 may preferentially augment IGF1R signaling in activated naive CD4⁺ T cells.

TABLE 2
Demographic information for subjects enrolled
in IGF1R signaling and proliferation studies.

| Cohort | Age | Sex | Ethnicity |
|---|---|---|---|
| IGF1R Signaling | 5 | F | Caucasian |
| | 5 | F | Caucasian |
| | 12 | M | Caucasian |
| | 13 | M | Caucasian |
| | 22 | F | Caucasian |
| | 23 | F | Caucasian |
| | 26 | M | Asian |
| | 27 | F | Asian |
| | 29 | F | Caucasian |
| Homeostatic Proliferation | 16 | F | African-American |
| | 18 | M | Caucasian |
| | 26 | F | Caucasian |
| | 41 | F | Caucasian |
| | 43 | F | Caucasian |

### Example 7. IGF1 Augments IL-2-Mediated Homeostatic Proliferation of Naïve Human Treg

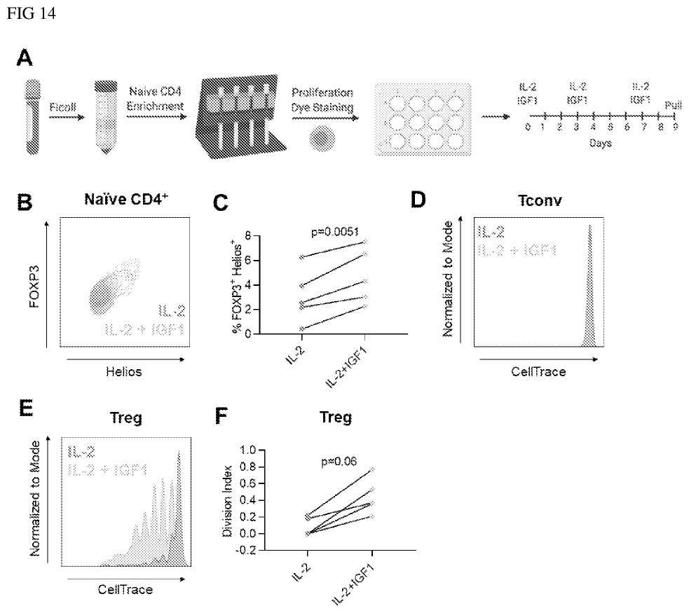

The observation that IGF1 augments TCR-mediated PI3K/Akt signaling in naïve CD4⁺ T cells implied that IGF1 could potentially synergize with IL-2 to preferentially induce the homeostatic proliferation of naïve human Tregs in the absence of TCR stimulation. Thus, bulk naïve CD4⁺ T cells were treated with IL-2 and/or IGF1 to assess Treg and Tconv proliferation (FIG. 14A, Table 3). Indeed, supplementation of low-dose IL-2 with IGF1 increased the percentage of FOXP3⁺Helios⁺ Tregs within bulk naïve CD4⁺ T cell culture (1.54-fold change, FIG. 14B-C). While low-dose IL-2+/−IGF1 did not induce the proliferation of naïve FOXP3⁻Helios⁻ Tconv (FIG. 14D), the addition of IGF1 promoted the expansion of naïve Tregs as compared to IL-2 alone (5.60-fold change, FIG. 14E-F). Thus, IGF1 can enhance the capacity of low-dose IL-2 to specifically drive the homeostatic proliferation of naïve Treg.

### Example 8. IGF1 and IL-2 Promote Treg Transduction While Maintaining Naivety

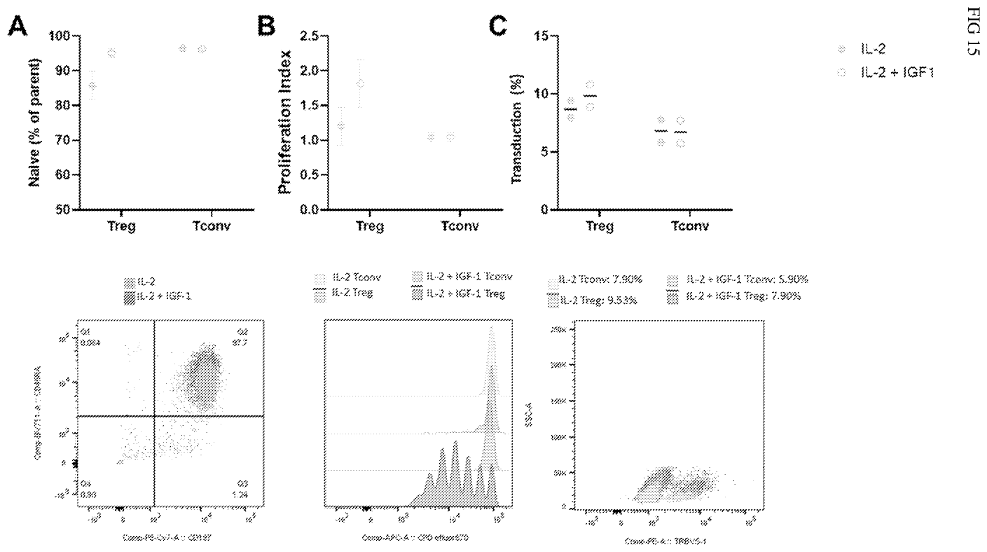

While maintenance of Treg naivety may eventually prove beneficial in certain translational uses such as quiescent Treg expansion for autologous adoptive cellular therapies, initially, the observations have informed the development of a novel methodology with basic science applications. The study of rare antigen-specific primary Treg responses has been classically hindered by the need for activation to permit transduction of a TCR of interest (38, 39). Thus, it was hypothesized that combinatorial IL-2 and IGF1 treatment would enable transduction of Tregs while avoiding acquisition of a memory phenotype. Indeed, pre-treatment of sorted Tregs with IL-2+IGF1 promoted a similar extent of proliferation as previously observed in bulk CD4⁺ T cell cultures (FIG. 15B, FIG. 14E-F), allowing for successful transduction (FIG. 15C). Importantly, Treg expressing the lentivirally-encoded T1D-associated TCR remained CD45RA⁺CD197⁺ naïve (FIG. 15A) as opposed to traditional methods that would generate memory antigen-specific Treg only. The resulting naïve antigen-specific Treg could then be utilized to study primary activation events, of particular interest in conditions such as T1D whereby many genetic risk loci are tagged to regulators of T cell activation (40, 41).

### REFERENCES

1. Zóka A, Műazes G, Somogyi A, Varga T, Szémán B, Al-Aissa Z, Hadarits O, Firneisz G: Altered immune regulation in type 1 diabetes. Clin Dev Immunol 2013; 2013:254874
2. Dejaco C, Duftner C, Grubeck-Loebenstein B, Schirmer M: Imbalance of regulatory T cells in human autoimmune diseases. Immunology 2006; 117:289-300
3. Zhao Y, Alard P, Kosiewicz M M: High Thymic Output of Effector CD4. J Immunol Res 2019; 2019:8785263
4. Brusko T, Wasserfall C, McGrail K, Schatz R, Viener H L, Schatz D, Haller M, Rockell J, Gottlieb P, Clare-Salzler M, Atkinson M: No alterations in the frequency of FOXP3+ regulatory T-cells in type 1 diabetes. Diabetes 2007; 56:604-612
5. Lindley S, Dayan C M, Bishop A, Roep B O, Peakman M, Tree T I: Defective suppressor function in CD4(+)CD25(+) T-cells from patients with type 1 diabetes. Diabetes 2005; 54:92-99
6. Putnam A L, Vendrame F, Dotta F, Gottlieb P A: CD4+CD25high regulatory T cells in human autoimmune diabetes. J Autoimmun 2005; 24:55-62
7. Ferraro A, Socci C, Stabilini A, Valle A, Monti P, Piemonti L, Nano R, Olek S, Maffi P, Scavini M, Secchi A, Staudacher C, Bonifacio E, Battaglia M: Expansion of Th17 cells and functional defects in T regulatory cells are key features of the pancreatic lymph nodes in patients with type 1 diabetes. Diabetes 2011; 60:2903-2913
8. Yu A, Snowhite I, Vendrame F, Rosenzwajg M, Klatzmann D, Pugliese A, Malek T R: Selective IL-2 responsiveness of regulatory T cells through multiple intrinsic mechanisms supports the use of low-dose IL-2 therapy in type 1 diabetes. Diabetes 2015; 64:2172-2183
9. Long S A, Rieck M, Sanda S, Bollyky J B, Samuels P L, Goland R, Ahmann A, Rabinovitch A, Aggarwal S, Phippard D, Turka L A, Ehlers M R, Bianchine P J, Boyle K D, Adah S A, Bluestone J A, Buckner J H, Greenbaum C J, Network DTatIT: Rapamycin/IL-2 combination therapy in patients with type 1 diabetes augments Tregs yet transiently impairs β-cell function. Diabetes 2012; 61:2340-2348
10. Booth N J, McQuaid A J, Sobande T, Kissane S, Agius E, Jackson S E, Salmon M, Falciani F, Yong K, Rustin M H, Akbar A N, Vukmanovic-Stejic M: Different proliferative potential and migratory characteristics of human CD4+ regulatory T cells that express either CD45RA or CD45RO. J Immunol 2010; 184:4317-4326
11. Vukmanovic-Stejic M, Zhang Y, Cook J E, Fletcher J M, McQuaid A, Masters J E, Rustin M H, Taams L S, Beverley P C, Macallan D C, Akbar A N: Human CD4+CD25hi Foxp3+ regulatory T cells are derived by rapid turnover of memory populations in vivo. J Clin Invest 2006; 116:2423-2433
12. Hoffmann P, Eder R, Boeld T J, Doser K, Piseshka B, Andreesen R, Edinger M: Only the CD45RA+ subpopulation of CD4+CD25high T cells gives rise to homogeneous regulatory T-cell lines upon in vitro expansion. Blood 2006; 108:4260-4267
13. Arroyo Hornero R, Betts G J, Sawitzki B, Vogt K, Harden P N, Wood K J: CD45RA Distinguishes CD4+CD25+CD127−/low TSDR Demethylated Regulatory T Cell Subpopulations With Differential Stability and Susceptibility to Tacrolimus-Mediated Inhibition of Suppression. Transplantation 2017; 101:302-309
14. Fritzsching B, Oberle N, Pauly E, Geffers R, Buer J, Poschl J, Krammer P, Linderkamp O, Sufi-Payer E: Naive regulatory T cells: a novel subpopulation defined by resistance toward CD95L-mediated cell death. Blood 2006; 108:3371-3378
15. Smith T J: Insulin-like growth factor-I regulation of immune function: a potential therapeutic target in autoimmune diseases? Pharmacol Rev 2010; 62:199-236
16. Shapiro M R, Atkinson M A, Brusko T M: Pleiotropic roles of the insulin-like growth factor axis in type 1 diabetes. Curr Opin Endocrinol Diabetes Obes 2019;
17. Bilbao D, Luciani L, Johannesson B, Piszczek A, Rosenthal N: Insulin-like growth factor-1 stimulates regulatory T cells and suppresses autoimmune disease. EMBO Mol Med 2014; 6:1423-1435
18. Kooijman R K, Scholtens L E, Rijkers G T, Zegers B J: Differential expression of type I insulin-like growth factor receptors in different stages of human T cells. Eur J Immunol 1995; 25:931-935
19. Shapiro M R, Wasserfall C H, McGrail S M, Posgai A L, Bacher R, Muir A, Haller M J, Schatz D A, Wesley J D, von Herrath M, Hagopian W A, Speake C, Atkinson M A, Brusko T M: Insulin-Like Growth Factor Dysregulation Both Preceding and Following Type 1 Diabetes Diagnosis. Diabetes 2019;
20. Johannesson B, Sattler S, Semenova E, Pastore S, Kennedy-Lydon T M, Sampson R D, Schneider M D, Rosenthal N, Bilbao D: Insulin-like growth factor-1 induces regulatory T cell-mediated suppression of allergic contact dermatitis in mice. Dis Model Mech 2014; 7:977-985
21. Budzinska M, Owczarz M, Pawlik-Pachucka E, Roszkowska-Gancarz M, Slusarczyk P, Puzianowska-Kuznicka M: miR-96, miR-145 and miR-9 expression increases, and IGF-1R and FOXO1 expression decreases in peripheral blood mononuclear cells of aging humans. BMC Geriatr 2016; 16:200
22. Palmer D B: The effect of age on thymic function. Front Immunol 2013; 4:316
23. Silva S L, Albuquerque A S, Serra-Caetano A, Foxall R B, Pires A R, Matoso P, Fernandes S M, Ferreira J, Cheynier R, Victorino R M, Caramalho I, Barata J T, Sousa A E: Human naïve regulatory T-cells feature high steady-state turnover and are maintained by IL-7. Oncotarget 2016; 7:12163-12175
24. DiToro D, Harbour S N, Bando J K, Benavides G, Witte S, Laufer V A, Moseley C, Singer J R, Frey B, Turner H, Bruning J, Darley-Usmar V, Gao M, Conover C, Hatton R D, Frank S, Colonna M, Weaver C T. Insulin-Like Growth Factors Are Key Regulators of T Helper 17 Regulatory T Cell Balance in Autoimmunity. Immunity. 2020; 52(4):650-67.e10. doi: 10.1016/j.immuni.2020.03.013. PubMed PMID: 32294406.
25. Bayer A L, Yu A, Malek T R. Function of the IL-2R for thymic and peripheral CD4+CD25+Foxp3+ T regulatory cells. Journal of immunology. 2007; 178(7):4062-71. PubMed PMID: 17371960.
26. Yu A, Zhu L, Altman N H, Malek T R. A low interleukin-2 receptor signaling threshold supports the development and homeostasis of T regulatory cells. Immunity. 2009; 30(2):204-17. PubMed PMID: 19185518.
27. Thornton A M, Korty P E, Tran D Q, Wohlfert E A, Murray P E, Belkaid Y, Shevach E M. Expression of Helios, an Ikaros transcription factor family member, differentiates thymic-derived from peripherally induced Foxp3+ T regulatory cells. J Immunol. 2010; 184(7):3433-41. Epub 2010/02/24. doi: 10.4049/jimmunol0904028. PubMed PMID: 20181882; PMCID: PMC3725574.
28. Thornton A M, Shevach E M. Helios: still behind the clouds. Immunology. 2019; 158(3):161-Epub 2019/10/13. doi: 10.1111/imm.13115. PubMed PMID: 31517385; PMCID: PMC6797934.
29. Kooijman R K, Scholtens L E, Rijkers G T, and Zegers B J. Differential expression of type I insulin-like growth factor receptors in different stages of human T cells. Eur J Immunol. 1995; 25(4):931-5.
30. Erlandsson M C, Töyrä Silfverswärd S, Nadali M, Turkkila M, Svensson M N D, Jonsson I M, et al. IGF-1R signalling contributes to IL-6 production and T cell dependent inflammation in rheumatoid arthritis. Biochim Biophys Acta Mol Basis Dis. 2017; 1863(9):2158-70.
31. Silva S L, Albuquerque A S, Serra-Caetano A, Foxall R B, Pires A R, Matoso P, et al. Human naïve regulatory T-cells feature high steady-state turnover and are maintained by IL-7. Oncotarget. 2016; 7(11):12163-75.
32. Bergerot I, Fabien N, Maguer V, and Thivolet C. Insulin-like growth factor-1 (IGF-1) protects NOD mice from insulitis and diabetes. Clin Exp Immunol. 1995; 102(2):335-40.
33. Cabello-Kindelan C, Mackey S, Sands A, Rodriguez J, Vazquez C, Pugliese A, et al. Immunomodulation Followed by Antigen-Specific T. Diabetes. 2020; 69(2):215-27.
34. Thornton A M, Korty P E, Tran D Q, Wohlfert E A, Murray P E, Belkaid Y, et al. Expression of Helios, an Ikaros transcription factor family member, differentiates thymic-derived from peripherally induced Foxp3+ T regulatory cells. J Immunol. 2010; 184(7):3433-41.
35. Thornton A M, and Shevach E M. Helios: still behind the clouds. Immunology. 2019; 158(3):161-70.
36. Budzinska M, Owczarz M, Pawlik-Pachucka E, Roszkowska-Gancarz M, Slusarczyk P, and Puzianowska-Kuznicka M. miR-96, miR-145 and miR-9 expression increases, and IGF-1R and FOXO1 expression decreases in peripheral blood mononuclear cells of aging humans. BMC Geriatr. 2016; 16(1):200.
37. Palmer D B. The effect of age on thymic function. Front Immunol. 2013; 4:316.
38. Yeh W I, Seay H R, Newby B, Posgai A L, Moniz F B, Michels A, et al. Avidity and Bystander Suppressive Capacity of Human Regulatory T Cells Expressing. Front Immunol. 2017; 8:1313.
39. Brusko T M, Koya R C, Zhu S, Lee M R, Putnam A L, McClymont S A, et al. Human antigen-specific regulatory T cells generated by T cell receptor gene transfer. PLoS One. 2010; 5(7):e11726.
40. Robertson C C, Inshaw J R J, Onengut-Gumuscu S, Chen W M, Flores Santa Cruz D, Yang H, et al. Fine-mapping, trans-ancestral and genomic analyses identify causal variants, cells, genes and drug targets for type 1 diabetes. bioRxiv. 2020:2020.06.19.158071.
41. Shapiro M R, Thirawatananond P, Peters L, Sharp R C, Ogundare S, Posgai A L, et al. Decoding genetic risk variants in type 1 diabetes. Immunol Cell Biol. 2021; 99(5):496-508.

What is claimed is:

1. A method for expansion of a naive lymphocyte population, the method comprising culturing the lymphocyte population under culture conditions that promote homeostatic expansion of the lymphocyte population, wherein the culture conditions comprise subjecting the population to growth factors IGF1 and common gamma chain.

2. The method of claim 1, wherein the expansion comprises cell proliferation.

3. The method of claim 2, wherein the subjecting step maintains a naïve phenotype of the lymphocyte population.

4. The method of claim 1, wherein the lymphocyte population originates from biological material source.

5. The method of any of claims 1-4, wherein the growth factor IGF1 is recombinantly produced, chemically synthesized, or purified from a biological sample.

6. The method of any of claims 1-5, wherein the common gamma chain is from IL2RG or CD132.

7. The method of claim 4, wherein the biological source is obtained from a subject or a donor subject.

8. The method of any of claims 1-7, wherein the lymphocyte population is cultured for about 9 to 11 days.

9. The method of claim 8, wherein the growth factors are replenished after 3 days.

10. The method of claims 8-9, wherein the growth factors are replenished after 7 days.

11. The method of claim 1, wherein the IGF1 has a concentration of about 100 ng/mL.

12. The method of claim 1, wherein the common gamma chain has a concentration of about 20 IU/mL.

13. The method of any of claims 1-12, further comprising administering the lymphocytes to the subject following the expansion.

14. A method for expansion of a naive regulatory T-Cell (Treg) population, the method comprising culturing the Treg population under culture conditions that promote homeostatic expansion of the Treg population, wherein the culture conditions comprise subjecting the population to growth factors IL-2 and IGF1.

15. The method of claim 14, wherein the expansion comprises cell proliferation.

16. The method of claim 15, wherein the subjecting step maintains a naïve phenotype of the Treg population.

17. The method of claim 14, wherein the Treg population originates from CD4⁺ T cells.

18. The method of claim 17, wherein the CD4⁺ T cells are obtained from biological material source by removing cells that contain CD8, CD14, CD16, CD19, CD20, CD25, CD36, CD56, CD61, CD66b, CD123, HLA-DR, TCRγ/δ, and glycophorin A from the biological material source and by selecting for cells that are CD4+ in the biological material source.

19. The method of claims 14-18, wherein the Treg population comprises naïve FOXP3⁺Helios⁺ regulatory T cells.

20. The method of any of claims 14-18, wherein the growth factors are recombinantly produced, chemically synthesized, or purified from a biological sample.

21. The method of any of claims 14-20, wherein the Treg population is cultured for about 9 to 11 days.

22. The method of claim 21, wherein the growth factors are replenished after 3 days.

23. The method of claim 21, wherein the growth factors are replenished after 7 days.

24. The method of claim 14, wherein the IGF1 has a concentration of about 100 ng/mL.

25. The method of claim 14, wherein the IL-2 has a concentration of about 20 IU/mL

26. The method of claim 18, wherein the biological source material is obtained from a subject or a donor subject.

27. The method of any of claims 14-26, further comprising administering the naïve Tregs to a subject following the expansion.

28. A method for modifying a lymphocyte population, comprising transducing one or more vectors into one or more cells of the lymphocyte population during or prior to culturing the lymphocyte population under culture conditions that promote homeostatic expansion of the naïve lymphocyte population, wherein the culture conditions comprise subjecting the population to growth factors IGF1 and common gamma chain.

29. The method of claim 28, wherein the method produces naïve lymphocytes containing the one or more vectors.

30. The method of claim 28, wherein the vectors encode a gene editing system engineered to reduce, prevent, increase, or otherwise modify expression of one or more gene products.

31. The method of claim 30, wherein the gene product is a T cell receptor or a chimeric antigen receptor.

32. The method of claim 30, wherein the gene editing system is selected from the group comprising a CRISPR-Cas 9 system, a lentiviral system, a zinc finger nuclease (ZFN) system, a Transcription Activator-Like Effector Nuclease (TALEN) system, or a meganuclease system.

33. The method of claim 32, wherein the gene editing system comprises a CRISPR-Cas 9 system.

34. The method of claim 32, wherein the gene editing system comprises a lentiviral system.

35. The method of claim 28, wherein the lymphocyte population is cultured in the growth factors for at least 7 days before transduction.

36. The method of claim 28, wherein the IGF1 has a concentration of about 100 ng/mL.

37. The method of claim 28, wherein the IL-2 has a concentration of about 20 IU/mL.

38. The method of claims 28-37, wherein the lymphocytes are administered to a subject or a recipient subject following the gene editing.

39. A method for treating autoimmune diseases in a subject in need, comprising administering IL-2 and IGF1 to the subject in need, wherein administering promotes homeostatic expansion of Tregs in vivo.

40. The method of claim 39, wherein the autoimmune disease comprises type 1 diabetes, multiple sclerosis, systemic lupus erythematosus, rheumatoid arthritis, Graves' disease, celiac disease, inflammatory bowel disease, or psoriasis.

41. The method of claim 39, wherein administration comprises oral, intranasal, parenteral (intravenous, intramuscular, intraperitoneal, or subcutaneous), rectal, or topical.

42. The method of any of claim 39, wherein IL-2 is administered every 3 days.

43. The method of claim 39, wherein IL-2 is co-administered with an anti-IL2 antibody.

44. The method of claim 39, wherein IGF1 is administered twice a day. The method of any of claims 39-44, wherein IGF1 has a dose of 20 μg/kg to 40 μg/kg.

46. The method of any of claims 39-44, wherein IL-2 has a dose of 0.09×10⁶ IU/m² to 0.47×10⁶ IU/m².
